(19) United States
(12) Patent Application Publication
(10) Pub. No.: US 2017/0182288 A1
(43) Pub. Date: Jun. 29, 2017

(54) DEVICES AND METHODS FOR INHIBITING OR PREVENTING COLONIZATION OF FLUID FLOW NETWORKS BY MICROORGANISMS

(71) Applicants: HOWARD A. STONE, PRINCETON, NJ (US); ZEMER GITAI, PRINCETON, NJ (US); MINYOUNG KEVIN KIM, PRINCETON, NJ (US); YI SHEN, ZURICH (CH); ALBERT SIRYAPORN, IRVINE, CA (US)

(72) Inventors: HOWARD A. STONE, PRINCETON, NJ (US); ZEMER GITAI, PRINCETON, NJ (US); MINYOUNG KEVIN KIM, PRINCETON, NJ (US); YI SHEN, ZURICH (CH); ALBERT SIRYAPORN, IRVINE, CA (US)

(73) Assignee: TRUSTEES OF PRINCETON UNIVERSITY, PRINCETON, NJ (US)

(21) Appl. No.: 15/386,402

(22) Filed: Dec. 21, 2016

Related U.S. Application Data
(60) Provisional application No. 62/272,535, filed on Dec. 29, 2015.

(51) Int. Cl. A61M 25/00; A61L 31/10; A61M 5/158; A61L 31/08

(52) U.S. Cl. CPC A61M 25/0045; A61L 31/08; A61L 31/10; A61M 5/158; A61L 2420/00; A61L 2400/18

(57) ABSTRACT

The invention includes novel devices and methods for inhibiting or preventing colonization of fluid flow networks by bacteria that have upstream surface motility. In certain aspects, the devices and methods of the invention prevent or minimize undesirable bacterial colonization of medical devices and/or treat or prevent bacterial infections.

### CROSS-REFERENCE TO RELATED APPLICATIONS

The present application claims priority under 35 U.S.C. §119(e) to U.S. Provisional Patent Application No. 62/272,535, filed Dec. 29, 2015, which is incorporated herein by reference in its entirety.

### STATEMENT REGARDING FEDERALLY SPONSORED RESEARCH OR DEVELOPMENT

This invention was made with government support under Grant No. OD-004389 awarded by the National Institutes of Health and Grant No. MCB-1330288 awarded by the National Science Foundation. The government has certain rights in the invention.

### BACKGROUND

Pseudomonas aeruginosa is an opportunistic pathogen that infects a broad range of hosts including plants and animals, In humans, it is a major cause of vascular-related illnesses including lung infections, urinary tract infections, bacteremia, and sepsis. In fluid flow environments, P. aeruginosa cells attach to surfaces using type IV pili (TFPs). These TFPs are localized to the bacterial cell poles, such that upon attaching to the surface, flow causes the bacteria to orient with the TFP pole pointed in the opposite direction of the flow. The repeated extension and retraction of TFPs in this position drives P. aeruginosa to move upstream along the surface. The upstream movement is a direct response to surface shear stress and is not due to chemotaxis. P. aeruginosa cells also swim through fluid environments using flagella, but upstream movement occurs without flagella.

While individual bacteria move upstream over short distances and small timescales, it is unknown whether populations can migrate physiological distances relevant for infection or colonize surfaces for extended periods in fluid flow. In particular, P. aeruginosa cells are dislodged from the surface by the fluid flow force and are subsequently pushed downstream. This finding raises a paradox: if cells are ejected from the surface and move backward along a flow streamline, the same streamline would carry cells back downstream upon detachment from the surface, nullifying any effect of the upstream movement. Furthermore, although individual cells move upstream, it is unknown whether this mechanism can drive the expansion of a multi-cellular population.

There is a need in the art for novel compositions, devices and methods for inhibiting or preventing colonization of fluid flow networks by microorganisms. In certain aspects, such methods and devices should allow for tuning the composition of multi-species bacterial communities in hosts, prevent or minimize inappropriate colonization in medical devices, and/or combat bacterial infections. The present invention meets this need.

### BRIEF SUMMARY OF THE INVENTION

The invention provides a fluid flow network comprising a fluid duct. The invention further provides a method of preventing or minimizing colonization of a fluid flow network comprising a fluid duct by a bacterium, wherein the fluid duct has a first opening and a second opening, wherein fluid can flow within the fluid duct from the first opening to the second opening, wherein the second opening comes into contact with the bacterium.

In certain embodiments, the fluid duct has a first opening and a second opening. In other embodiments, fluid can flow within the fluid duct from the first opening (upstream) to the second opening (downstream). In yet other embodiments, the second opening can come into contact with a bacterium. In yet other embodiments, at least a portion of the internal surface of the fluid duct is derivatized with a coating that increases adhesion of the bacterium to the internal surface of the fluid duct as compared to the underivatized portion of the internal surface of the fluid.

In certain embodiments, at least a portion of the internal surface of the fluid duct in proximity of at least one selected from the group consisting of the first opening and the second opening is derivatized with a coating that increases adhesion of the bacterium to the internal surface of the fluid duct as compared to the underivatized portion of the internal surface of the fluid.

In certain embodiments, essentially the entire internal surface of the fluid duct is derivatized with a coating that increases adhesion of the bacterium to the internal surface of the fluid duct as compared to the underivatized internal surface of the fluid.

In certain embodiments, the bacterium comprises an upstream surface migrating bacterium. In other embodiments, the bacterium comprises Pseudomonas aeruginosa. In yet other embodiments, the bacterium comprises at least one selected from the group consisting of Neisseria gonorrhoeae, Neisseria meningitidis, Legionella pneumophila, and Streptococcus sanguinis. In yet other embodiments, the bacterium comprises at least one selected from the group consisting of Acinetobacter calcoaceticus, Aeromonas hydrophila, Azoarcus spp., Bacteroides ureolyticus, Branhamella catarrhalis, Comomonas testosterone, Dichelobacter nodosus, Eikenella corrodens, Kingella denitrificans, Kingella kingae, Legionella pneumophila, Moraxella bovis, Moraxella lacunata, Moraxella nonliquefaciens, Moraxella kingie, Mycoplasma mobile, Pseudomonas stutzeri, Pseudomonas putida, Pseudomonas syringae, Ralstonia solanacearum, Shewanella putrefaciens, Suttonella indologenes, Vibrio cholera, Wolinella spp., and/or Xylella fastidiosa.

In certain embodiments, derivatization of at least a portion of the internal surface of the fluid duct in proximity of the second opening with a coating that increases adhesion of the bacterium to the internal surface of the fluid duct prevents or minimizes migration, or reduces the migration rate, of the bacterium through the fluid duct towards the first opening.

In certain embodiments, the duct is part of at least one selected from the group consisting of a catheter, intravenous line and needle.

In certain embodiments, the fluid flowing through the duct comprises a bodily fluid. In other embodiments, the bodily fluid comprises at least one selected from the group consisting of blood, serum, plasma and urine.

In certain embodiments, at least one selected from the group consisting of the first opening and second opening is in proximity to a branching point of the network, wherein the fluid duct is in fluid communication with one or more other fluid ducts.

In certain embodiments, at least a portion of the internal surface of the fluid duct in proximity to the branching point of the network is derivatized with a coating that increases adhesion of the bacterium to the internal surface of the fluid duct as compared to the underivatized portion of the internal surface of the fluid.

In certain embodiments, the coating comprises at least one chemical group selected from the group consisting of an alcohol and a thiol. In other embodiments, the coating comprises at least one protein selected from the group consisting of fibronectin, fibrin and fibrinogen. In yet other embodiments, the internal surface of the fluid duct comprises at least one selected from the group consisting of silica or glass, and wherein the coating comprises at least one silane. In yet other embodiments, the fluid flow network of the invention is part of an organism's vasculature. In yet other embodiments, the fluid duct is an implantable device that is implanted within the organism's vasculature. In yet other embodiments, the organism comprises at least one selected from the group consisting of a plant and an animal.

In certain embodiments, the method comprises derivatizing at least a portion of the internal surface of the fluid duct with a coating that increases adhesion of the bacterium to the internal surface of the fluid duct as compared to the underivatized portion of the internal surface of the fluid.

In certain embodiments, at least a portion of the internal surface of the fluid duct in proximity of at least one selected from the group consisting of the first opening and the second opening is derivatized with a coating that increases adhesion of the bacterium to the internal surface of the fluid duct as compared to the underivatized portion of the internal surface of the fluid.

In certain embodiments, essentially the entire internal surface of the fluid duct is derivatized with a coating that increases adhesion of the bacterium to the internal surface of the fluid duct as compared to the underivatized internal surface of the fluid.

In certain embodiments, the bacterium comprises an upstream surface migrating bacterium. In other embodiments, the bacterium comprises Pseudomonas aeruginosa. In yet other embodiments, the bacterium comprises at least one selected from the group consisting of Neisseria gonorrhoeae, Neisseria meningitidis, Legionella pneumophila, and Streptococcus sanguinis. In yet other embodiments, the bacterium comprises at least one selected from the group consisting of Acinetobacter calcoaceticus, Aeromonas hydrophila, Azoarcus spp., Bacteroides ureolyticus, Branhamella catarrhalis, Comomonas testosterone, Dichelobacter nodosus, Eikenella corrodens, Kingella denitrificans, Kingella kingae, Legionella pneumophila, Moraxella bovis, Moraxella lacunata, Moraxella nonliquefaciens, Moraxella kingie, Mycoplasma mobile, Pseudomonas stutzeri, Pseudomonas putida, Pseudomonas syringae, Ralstonia solanacearum, Shewanella putrefaciens, Suttonella indologenes, Vibrio cholera, Wolinella spp., and/or Xylella fastidiosa.

In certain embodiments, derivatizing the internal surface of the fluid duct in proximity of the second opening with a coating that increases adhesion of the bacterium to the internal surface of the fluid duct prevents or minimizes migration, or reduces the migration rate, of the bacterium through the fluid duct towards the first opening.

In certain embodiments, the duct is part of at least one selected from the group consisting of a catheter, intravenous line and needle.

In certain embodiments, the fluid flowing through the duct comprises a bodily fluid. In other embodiments, the bodily fluid comprises at least one selected from the group consisting of blood, serum, plasma and urine.

In certain embodiments, at least one selected from the group consisting of the first opening and second opening is in proximity to a branching point of the network, wherein the fluid duct is in fluid communication with one or more other fluid ducts.

In certain embodiments, at least a portion of the internal surface of the fluid duct in proximity to the branching point of the network is derivatized with a coating that increases adhesion of the bacterium to the internal surface of the fluid duct as compared to the underivatized portion of the internal surface of the fluid.

In certain embodiments, the coating comprises at least one chemical group selected from the group consisting of an alcohol and a thiol.

In certain embodiments, the coating comprises at least one protein selected from the group consisting of fibronectin, fibrin and fibrinogen.

In certain embodiments, the internal surface of the fluid duct comprises at least one selected from the group consisting of silica or glass, and wherein the coating comprises at least one silane.

### BRIEF DESCRIPTION OF THE FIGURES

For the purpose of illustrating the invention, there are depicted in the drawings certain embodiments in accordance with the present invention. However, the invention is not limited to the precise arrangements and instrumentalities of the embodiments depicted in the drawings.

FIG. 1 is a non-limiting illustration of upstream dispersal of P. aeruginosa populations in flow networks. Panel A: Schematic summarizing upstream movement by P. aeruginosa. Fluid flow orients polar-attached cells, such that the attachment pole is positioned upstream and pilus retraction moves cells upstream. Panel B: P. aeruginosa surface colonization in a linear microfluidic channel. Cells are loaded behind a “start line” in which cell-free medium flows from the left to right. Within 2 hours, cells at the leading edge of the population reach the “finish line.” Panel C: Vascular flow networks in chicken embryos and E. aureum plant leaves contain many branching intersections (arrows). Panel D: Schematic of a branched flow network microfluidic device used to track colonization, competition, and dispersal. Cell-free medium flowing through the main branch diverges into two branches. Cells are inoculated into the “seeded branch.” Arrows indicate the direction of flow. Panel E: Tracking of fluorescent micro-tracer beads indicates a flow pattern that is laminar, stable, and uni-directional. Scale bars represent 2 mm in Panel C and 100 μm in Panel E.

FIG. 2 is a non-limiting illustration of the finding that upstream dispersal provides P. aeruginosa with a selective growth advantage in flow. Panel A: Surface colonization of wild-type (green) and pilus-defective ΔpilTU (red) P. aeruginosa cells in a branched network. An equal number of each strain was initially co-inoculated into the seeded branch. After 15 hours, wild-type cells colonized all areas of the device, whereas ΔpilTU cells remained in the seeded branch. The effluent (containing planktonic and surface-detached cells) from each branch was collected and cultured for an additional 3.5 hours. Panels B-D: The total number of cells in each effluent was measured through optical density (Panel B) and the effluent population composition was determined using fluorescence microscopy (Panels C-D). ΔpilTU cells were not detected (ND) in the side branch. Error bars in Panel D indicate the SD. Panel E: Trajectories of individual wild-type cells on surfaces, which move upstream (green) or downstream (cyan) in “zigzag” paths that cross laterally into different streamlines. Each trajectory was acquired at 30 second intervals for 30 min. Scale bars indicate 100 μm in Panels A and E, and 5 μm in Panel C. All experiments were performed in triplicate.

FIG. 3 is a non-limiting illustration of the finding that pathogens self-segregate to co-exist in a branched flow network. Panel A: Surface colonization of P. aeruginosa (green) and P. mirabilis (red) cells in a branched network. Both strains were initially co-inoculated in equal numbers into the seeded branch. After 15 hours of continuous flow, P. aeruginosa colonized the upper, seeded, and side branches, whereas P. mirabilis colonized only the seeded branch. The dashed box (top) shows a region that is 100 μm upstream from the indicated section. Panels B- D: The effluent (containing planktonic and surface-detached cells) from each branch was analyzed for (Panel B) total cell number using optical density and (Panels C-D) population composition using fluorescence microscopy. P. mirabilis was the dominant species in the seeded branch, whereas P. aeruginosa was the only species that colonized the side branch. ND indicates cells were not detected. Error bars in Panel D indicate the SD. Scale bars in Panels A and C indicate 100 μm and 5 respectively. Experiments were performed in triplicate.

FIG. 4 is a non-limiting illustration of exemplary theoretical and natural host models of upstream dispersal. Panel A: Schematic illustrating the diverse motility modes observed during upstream dispersal. Panel B: Surface colonization of wild-type P. aeruginosa after 15 hours of flow in untreated and thiol-treated linear channels. Panel C: Corresponding population density data for untreated (circles) or thiol-treated (boxes) devices and population densities predicted by our upstream dispersal model (lines). Panel D: Schematic depicting the mechanism of upstream dispersal. (i) Cells move upstream in a zigzag trajectory on surfaces, enter side branch streamlines, detach from the surface, and are carried downstream by the flow. (ii) Time evolution of a surface population that advances toward a branching intersection through counter-advection and lateral diffusion. Panel E: Prediction from the model that the number of cells entering side-branch streamlines increases exponentially with time. Panel F: Surface colonization of wild-type (green) and surface-motility-defective ΔpilTU (red) P. aeruginosa cells in a thiol-treated device after 15 hours of continuous flow. No cells were observed in the side channel (see FIG. 8, Panel A). Panel G: Plant colonization assay in which a tobacco plant leaf was inoculated with equal numbers of wild-type and ΔpilTU P. aeruginosa cells. After 7 days, wild-type cells (green) were observed in the upstream vasculature, whereas ΔpilTU cells (red) were found at the inoculation site. Panel H: Schematic showing a generalized branched flow network (gray) with characteristic pore spacing 1 and colonization of the network by bacterial communities (black) that migrate upstream a distance 1. Scale bars represent 100 μm in Panel F and 5 mm in Panel G.

FIG. 5 is a set of images and graphs illustrating swimming motility of ΔpilTU cells, planktonic growth competition between wild-type and ΔpilTU strains, and the sizes of cells from the seeded and side branches of branched flow networks, related to FIG. 1. Panel A: Swimming motility of wild-type, ΔpilTU , and flgK P. aeruginosa strains on 0.3% agar plates. Panel B: Planktonic growth competitions between wild-type and ΔpilTU P. aeruginosa strains that were co-cultured at mid-exponential phase in culture tubes at 37° C. for 3.5 hours and analyzed using fluorescence microscopy. Panel C: Comparison of wild-type cell sizes in the effluents of seeded and side branches (containing planktonic and surface-detached cells) that were collected during 15 hours of continuous flow, incubated at 37° C. for 3.5 hours and imaged using phase contrast and fluorescence microscopy (see FIG. 2, panel C). Wild-type cells were co-inoculated with ΔpilTU cells in equal numbers into the seeded branch. For panels B-C, at least 200 cells were analyzed in each experiment. Bars are the average of three independent experiments and error bars indicate standard deviation.

FIG. 6 is a set of images illustrating upstream dispersal in a converging flow network, arrangement of type IV pili at the cell pole, and competition between P. aeruginosa and E. coli or B. subtilis, related to FIG. 2. Panel A: Upstream surface colonization in a converging branched flow network. Arrows indicate the directions that cell-free medium and bacterial cells were flowed. Wild-type (green) and ΔpilTU (red) P. aeruginosa cells were initially seeded downstream of the converging channels. Wild-type cells reached all upstream channels by 17 hours while the ΔpilTU cells remained in the downstream regions. Panel B: Transmission electron microscopy image of type IV pili (white), which extend radially from the cell body pole (red) in a pilT mutant. (Panel C; upper portion) E. coli (yellow) or (Panel D; upper portion) B. subtilis (black) on the surface of diverging branched networks after 15 hours of continuous flow. Strains were co-inoculated into the seeded branch with an equal number of surface motility-defective (ΔpilTU , red) P. aeruginosa cells. Note the dark spots in the lower left corner of Panel D are debris from the device fabrication process and that few B. subtilis colonized the surface of the seeded branch. (Lower portions of Panels C-D) The effluent from each branch (containing planktonic and surface-detached cells) was collected and grown on agar plates overnight. Lawns of bacteria were observed in the effluents of seeded branches and no growth was observed in the effluents of side branches. Scale bars represent 50 μm for panel A, 0.5 μm for Panel B, and 100 μm for Panels C-D.

FIG. 7 is a series of images and graphs illustrating non-limiting P. mirabilis growth and surface motility rates, planktonic growth competition between P. mirabilis and P. aeruginosa, comparison of P. aeruginosa cell sizes from seeded and side branches in competition experiments with P. mirabilis, and flow competition experiments between P. aeruginosa and S. aureus or S. Typhimurium, related to FIG. 3. Panel A: Growth rates of P. aeruginosa and P. mirabilis at mid-exponential phase in separate culture tubes at 37° C. Panel B: Planktonic growth competitions indicating the relative composition of P. aeruginosa and P. mirabilis in mid-exponential cultures that were mixed, grown in culture tubes for 3.5 hours, and analyzed using fluorescence microscopy. Panel C: The surface motility velocity of P. aeruginosa and P. mirabilis cells, which were stabbed through 0.75% agar pads and imaged at the cover-glass surface. Error bars indicate standard error. Panel D: Comparison of the cell sizes for wild-type P. aeruginosa in the effluents of seeded and side branches (containing planktonic and surface-detached cells) that were collected during 15 hours of continuous flow, incubated at 37° C. for 3.5 hours, and imaged using phase contrast and fluorescence microscopy (see FIG. 3C). P. mirabilis and P. aeruginosa were co-inoculated in equal numbers into the seeded branch. At least 200 cells were analyzed in each experiment. Panel E: Surface colonization of P. aeruginosa (green) and S. aureus (black, arrow pointing to several cells) cells after 12.5 hours of continuous flow. Equal numbers of each strain were co-inoculated into the seeded branch. Panel F: Surface colonization of P. aeruginosa and S. Typhimurium in a (i) T-shaped device, (ii) Equal numbers of P. aeruginosa (green) and S. Typhimurium (black) were co-inoculated into the channel, (iii) Upstream colonization of P. aeruginosa after 48 hours of continuous flow. Bars in Panels A-B and D are the average of three independent experiments and error bars indicate standard deviation. Scale bars represent 100 μm in Panel E and 50 μm in Panel F.

FIG. 8 is a series of images and graphs illustrating number of P. aeruginosa cells detected in thiol-coated devices, and P. aeruginosa colonization in plant vasculature, related to FIG. 4. Panel A: Total number of cells in the effluents (containing planktonic and surface-detached cells) of the seeded and side branches of thiol-coated devices (see FIG. 4, Panel F). The effluent from each branch was collected during 15 hours of continuous flow, cultured for 3.5 hours at 37° C., and measured for optical density. Bacterial cells were not detected (ND) in the side branch. Panels B-C: P. aeruginosa colonization in a tobacco plant leaf. Wild-type and ΔpilTU cells were co-inoculated in equal numbers into the leaf using syringe infiltration. After 7 days, wild-type (green) cells were found in the upstream vasculature while ΔpilTU cells (red) were found only at the inoculation site. Scale bars represent 5 mm. Panel D: (i) Schematic depicting the inoculation zone (green) and the encompassing region containing nearby vascular branches that were used for image analysis. The upstream region was defined as the encompassing region excluding the inoculation zone. (ii) The competitive indexes between wild-type and ΔpilTU cells in each region were computed by taking the ratio of the GFP and mCherry fluorescence intensities.

FIG. 9 illustrates a non-limiting aspect of a model of bacterial dispersal by upstream migration. Panel A: Schematic of a non-limiting experimental setup used to characterize upstream dispersal. Cells are initially confined behind a start line and migrate upstream towards the finish line. New cells are continuously seeded behind the start line from the source. Panel B: Schematic indicating steps involved in a process of upstream dispersal.

FIG. 10 is a graph illustrating distribution of instantaneous P. aeruginosa upstream velocities determined in Shen, et al., 2012, Biophys. J. 103:146-151, which is incorporated herein in its entirety by reference.

FIG. 11 is a graph illustrating non-limiting experimental data indicating the population density in untreated (black points) and thiol-treated (gray points) channels after 17 hours of continuous flow. Equation (16) is plotted for (α−β, μ₀)=(0.12/hr, 30) (black line) and (α−β, μ₀)=(0.19/hr, 1550) (gray line).

FIG. 12 is a set of images and graphs illustrating upstream migration in a branched flow network. Panel A: Schematic indicating the movement of a bacterial population that spreads out laterally as it moves upstream (towards −∞ along the x-axis). The population reaches the branch junction at t=2. Panel B: Plot of the population density along the y-axis (Equation (19) at t₂ and at t₄.

### DETAILED DESCRIPTION OF THE INVENTION

The present invention provides novel compositions, devices and methods for inhibiting or preventing colonization of fluid flow networks by microorganisms. The present invention relates, in one aspect, to the discovery that certain bacteria can colonize fluid flow networks by undergoing upstream surface migration. In certain embodiments, the upstream surface migration for the bacteria contemplated within the invention is higher in magnitude than any downstream pushing caused by fluid flow dislodging the bacteria from the walls of the fluid flow networks, thus resulting in net upstream movement of the bacteria.

Pseudomonas aeruginosa is an important human pathogen that is a major cause of sepsis, bacteremia, hospital-acquired infections, burn wound infections, and cystic fibrosis infections. This bacterium causes infections by entering the fluid networks of the body, where it can spread and grow. As demonstrated herein, P. aeruginosa communities use a diverse set of motility strategies, including a novel surface-motility mechanism characterized by counter-advection and transverse diffusion, to rapidly disperse throughout vasculature-like flow networks. These motility modalities give P. aeruginosa a selective growth advantage, enabling it to self-segregate from other human pathogens such as Proteus mirabilis and Staphylococcus aureus that outcompete P. aeruginosa in well-mixed non-flow environments. A quantitative model of bacterial colonization in flow networks was developed and confirmed in an in vivo study in plant vasculature. In certain non-limiting embodiments, colonization and/or dispersal can be inhibited by modifying surface chemistry. The present results show that the interaction between flow mechanics and motility structures shapes the formation of mixed-species communities and suggest a general mechanism by which bacteria could colonize hosts. Furthermore, the present results suggest novel strategies for tuning the composition of multi-species bacterial communities in hosts, preventing inappropriate colonization in medical devices, and combatting bacterial infections.

In certain embodiments, the bacteria contemplated within the invention have at least one pilus, which allows for upstream surface migration. In other non-limiting embodiments, the bacteria have at least one type IV pilus. In certain non-limiting examples, a bacterium aligns itself with the stream, so that the at least one pilus is aligned with the stream and pointing upstream. Retraction of the at least one pilus promotes cell migration upstream along the surface. In certain embodiments, such bacteria are referred to as upstream surface migrating bacteria.

Embodiments of the present invention can be used to inhibit the spread and growth of upstream surface migrating bacteria in fluid flow networks, thereby reducing the possibility of infection caused by these devices when they are contacted with the vasculature of a living organism, such as a plant or animal, such as a human. In certain embodiments, the present compositions and methods inhibit bacterial entry into the organism's vasculature at wound sites and/or prevent bacterial spreading and growth in vasculature networks. In other embodiments, the present compositions and methods prevent or reduce sepsis and/or other vasculature infections in the organism.

One illustrative example of a bacterium contemplated within the invention comprises Pseudomonas aeruginosa (P. aeruginosa). As disclosed herein, P. aeruginosa uses a complex combination of motility modes to spread within fluid flow networks. For example, P. aeruginosa uses specialized surface motility mechanisms to move upstream, laterally and downstream, enabling it to spread through and colonize all parts of a fluid network. This motility allows P. aeruginosa to explore and take over nutrient-rich environments, giving it a significant growth advantage over other bacteria.

Additional illustrative examples of bacteria contemplated within the invention comprise Neisseria gonorrhoeae, Neisseria meningitidis, Legionella pneumophila, and/or Streptococcus sanguinis. Additional illustrative examples of bacteria contemplated within the invention comprise Acinetobacter calcoaceticus, Aeromonas hydrophila, Azoarcus spp., Bacteroides ureolyticus, Branhamella catarrhalis, Comomonas testosterone, Dichelobacter nodosus, Eikenella corrodens, Kingella denitrificans, Kingella kingae, Legionella pneumophila, Moraxella bovis, Moraxella lacunata, Moraxella nonliquefaciens, Moraxella kingie, Mycoplasma mobile, Pseudomonas stutzeri, Pseudomonas putida, Pseudomonas syringae, Ralstonia solanacearum, Shewanella putrefaciens, Suttonella indologenes, Vibrio cholera, Wolinella spp., and/or Xylella fastidiosa.

The invention includes methods for inhibiting colonization, growth and dispersal of pathogenic bacteria (such as upstream surface migrating bacteria) in a fluid flow network through surface chemistry treatments and/or fluid flow control. The fluid flow network can comprise any fluid duct that can deliver a fluid stream and may enter in contact with an upstream surface migrating bacterium. Non-limiting examples of such ducts include healthcare devices, such as catheter, intravenous lines and needles, all manners of fluid distribution systems in fluid-handling systems or small devices, and an organism's vasculature, such as the vasculature exposed to an external puncture due to insertion of a catheter or needle and/or disrupted by a wound and/or injury.

In certain aspects, at least a portion of the internal surface of the fluid duct is chemically and/or physically modified with a coating that increases the adhesion of the contemplated bacterium to the portion of the internal surface of the fluid duct. In other embodiments, the coated portion creates a bacterial “sticky trap,” which prevents or minimizes mobilization of the bacteria upstream.

In certain embodiments, the coated portion of the internal surface of the fluid duct is located upstream from the position where the upstream surface migrating bacteria may penetrate the fluid duct. In other embodiments, the coated portion of the internal surface of the fluid duct is located upstream from, and in contact with and/or in near proximity to, the position where the upstream surface migrating bacteria may penetrate the fluid duct. In yet other embodiments, the coated portion of the internal surface of the fluid duct is located upstream from the position where the upstream surface migrating bacteria may penetrate the fluid duct, and in contact with and/or in near proximity to a branching point of the fluid duct. The branching point of the fluid duct is defined as the position where the fluid duct of interest is connected with one or more other fluid ducts, such as the fluid duct of interest and the one or more other fluid ducts are in fluid communication. In certain embodiments, presence of the coated portion of the internal surface of the fluid duct avoids or minimizes colonization of the other one or more ducts by the bacteria.

In certain embodiments, chemical and/or physical modification of the internal surface of the fluid duct can be achieved using any methods known in the art. In an illustrative example, the internal surface of the fluid duct is derivatized with an agent that contains a chemical group selected from the group consisting of a thiol and a hydroxyl. The derivatization can be achieved using methods known in the art. In an illustrative example, if the internal surface of the fluid duct comprises glass or silica, the agent can be a silane comprising a free (or masked) thiol and/or hydroxyl, and further comprising a reactive group capable of reacting with the silica and/or glass of the internal surface of the fluid. Such reactive groups can be, for example, chloride, bromide, iodide and/or silane (such as, but not limited to, a monoalkoxysilane, a dialkoxysilane and/or a trialkoxysilane). In another illustrative example, the agent comprises a protein, such as fibronectin, fibrin and/or fibrinogen, derivatized with a linker (such as, but not limited to, a linker comprising a reactive group, such as but not limited to a chloride, bromide, iodide or silane, such as but not limited to a monoalkoxysilane, a dialkoxysilane and/or a trialkoxysilane), wherein the chemical reagent is capable of attaching the protein to the internal surface of the fluid duct.

In other aspects, the invention contemplates controlling flow patterns in a fluid flow network such that the fluidic shear stresses inhibit upstream and lateral motility of any upstream surface migrating bacteria present in the network. In certain embodiments, this is achieved by designing the geometry and architecture of flow networks and/or by controlling the rate at which fluid is flowed through networks, so as to maximize shear stress at critical regions and at potential sites of infection. In other embodiments, such design allows for sweeping surface-attached bacteria off the internal surface of the network and downstream, thereby preventing upstream and lateral spreading of the bacteria. In yet other embodiments, distances between branches of flow networks are maximized to minimize and/or eliminate the effects of upstream migration.

The present invention further provides fluid flow networks prepared using the methods recited herein. Fluid flow networks in accordance with the present invention can be used, for example, for manipulating healthcare fluids (such as medications, saline, plasma, blood, serum, urine and so forth), food industry fluids (such as water, beverages, ingredients and so forth), or any other fluid that can enter in contact with upstream surface migrating bacteria.

In certain embodiments, the fluid flow networks contemplated within the invention include an organism's vasculature. In other embodiments, the organism's vasculature and/or body may be invaded and/or colonized by upstream surface migrating bacteria that penetrate the vasculature due to an external puncture due to insertion of a catheter or needle into the vasculature. In yet other embodiments, the organism's vasculature and/or body may be invaded and/or colonized by upstream surface migrating bacteria that penetrate the vasculature through a wound and/or injury to the organism. In yet other embodiments, colonization of the vasculature and/or body of the organism by an upstream surface migrating bacterium can be minimized or prevented by coating a portion of the internal surface of the organism's vasculature that is located upstream from the puncture, wound and/or injury with a composition that increases adhesion of the bacterium to the internal surface of the organism's vasculature. Such composition is selected, so that it can attach covalently and/or physically to the internal surface of the organism's vasculature and displays a chemical group (such as, but not limited to, an hydroxyl and/or thiol) and/or a protein (such as, but not limited to, fibronectin, fibrin and/or fibrinogen) that adheres to the bacterium. In yet other embodiments, the adhesion increasing composition is attached chemically and/or physically to an implantable device (such as, but not limited to, a flat or curved surface, a fluid duct section, and the like), which is then implanted within the patient's vasculature at a position that is upstream from the puncture, wound and/or injury. In yet other embodiments, the implantable device is removed from the organism's vasculature once the risk of bacterial colonization in the organism's vasculature is minimized or ruled to be absent. In yet other embodiments, the implantable device is not removed from the vasculature once the risk of bacterial colonization in the patient's vasculature is minimized or ruled to be absent. In yet other embodiments, the implant is at least partially biodegradable.

Definitions

As used herein, each of the following terms has the meaning associated with it in this section.

As used herein, unless defined otherwise, all technical and scientific terms generally have the same meaning as commonly understood by one of ordinary skill in the art to which this invention belongs. Generally, the nomenclature used herein and the laboratory procedures in surface chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “fluid” refers to a homogeneous or heterogeneous phase that is capable of demonstrating fluidic (flowing) behavior under the experimental conditions under consideration. In certain embodiments, a fluid comprises a liquid or a gas. In other embodiments, the fluid consists essentially of a liquid or a gas. In yet other embodiments, the fluid consists of a liquid or a gas. In yet other embodiments, a fluid comprises a liquid. In yet other embodiments, the fluid consists essentially of a liquid. In yet other embodiments, the fluid consists of a liquid.

As used herein, the term “instructional material” includes a publication, a recording, a diagram, or any other medium of expression that may be used to communicate the usefulness of the compositions, devices and/or methods of the present invention. In certain embodiments, the instructional material may be part of a kit useful for generating compositions of the present invention. The instructional material of the kit may, for example, be affixed to a container that contains compositions and/or devices of the present invention or be shipped together with a container that contains compositions and/or devices of the present invention. Alternatively, the instructional material may be shipped separately from the container with the intention that the recipient uses the instructional material and compositions, methods and/or devices cooperatively. For example, the instructional material is for use of a kit; or instructions for use of the compositions, methods and/or devices of the present invention.

As used herein, the term “μm” is the abbreviation for “micron” or “micrometer”, and it is understood that 1 μm=0.001 mm=10⁻⁶ m=1 millionth of a meter.

As used herein, the term “nm” is the abbreviation for “nanometer” and it is understood that 1 nm=1 nanometer=10⁻⁹ m=1 billionth of a meter.

As used herein, the term “pilus” (plural form being pili) refers to a hair like appendage found on the surface of many bacteria. Some pili, called type IV pili, generate motile forces. In certain embodiments, the external ends of the pili adhere to a solid substrate, either the surface to which the bacterium is attached or to other bacteria, and when the pilus contracts, it pulls the bacterium forward, like a grappling hook. Movement produced by type IV pili is typically called twitching motility, as distinct from other forms of bacterial motility, such as that produced by flagella. However, some bacteria, for example Myxococcus xanthus, exhibit gliding motility.

As used herein, the term “TFP” refers to type IV pilus and/or pili.

As used herein, the phrase “upstream surface migrating bacterium” refers to a bacterium that is capable of migrating upstream along the inner surface of a fluidic flow network. In certain embodiments, the bacterium has one or more pilus/pili. In other embodiments, the bacterium has one or more type IV pilus/pili. In yet other embodiments, the bacterium is pathogenic. In yet other embodiments, non-limiting examples of upstream surface migrating bacteria comprise Pseudomonas aeruginosa, Neisseria gonorrhoeae, Neisseria meningitidis, Legionella pneumophila, and/or Streptococcus sanguinis. In yet other embodiments, non-limiting examples of upstream migrating bacteria comprise Acinetobacter calcoaceticus, Aeromonas hydrophila, Azoarcus spp., Bacteroides ureolyticus, Branhamella catarrhalis, Comomonas testosterone, Dichelobacter nodosus, Eikenella corrodens, Kingella denitrificans, Kingella kingae, Legionella pneumophila, Moraxella bovis, Moraxella lacunata, Moraxella nonliquefaciens, Moraxella kingie, Mycoplasma mobile, Pseudomonas stutzeri, Pseudomonas putida, Pseudomonas syringae, Ralstonia solanacearum, Shewanella putrefaciens, Suttonella indologenes, Vibrio cholera, Wolinella spp., and/or Xylella fastidiosa.

Throughout this disclosure, various aspects of the present invention may be presented in a range format. It should be understood that the description in range format is merely for convenience and brevity and should not be construed as an inflexible limitation on the scope of the present invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6, and so on, as well as individual numbers within that range, for example, 1, 2, 2.7, 3, 4, 5, 5.3, and 6. This applies regardless of the breadth of the range.

Disclosure

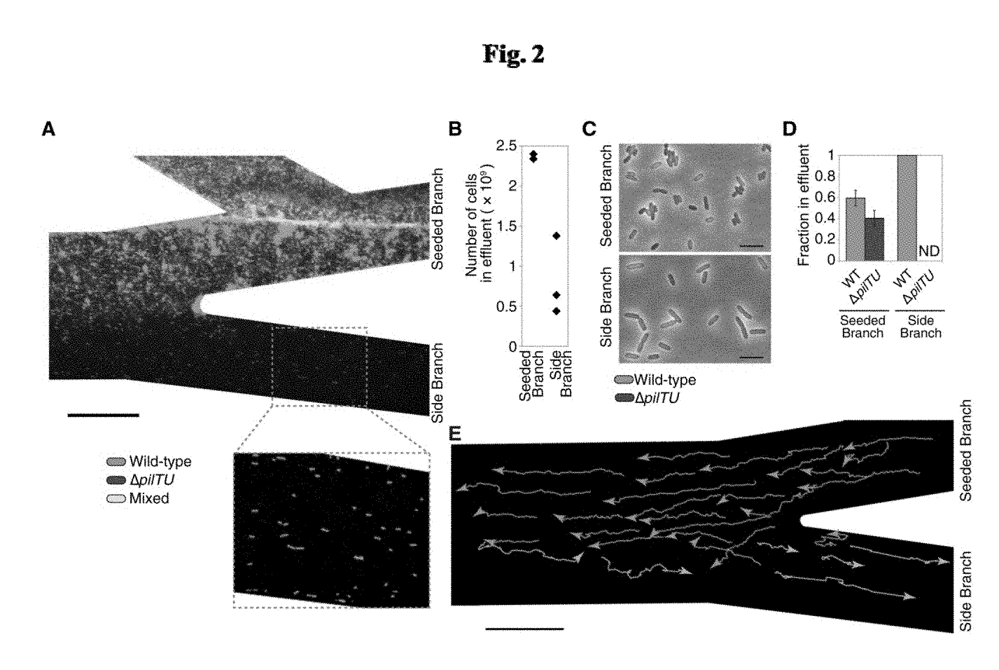

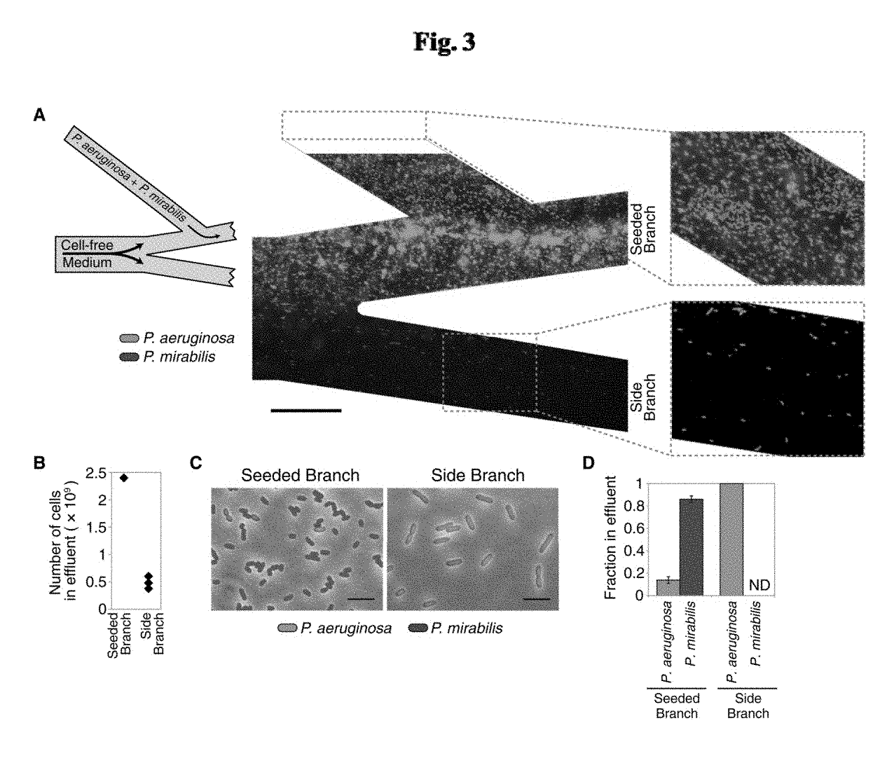

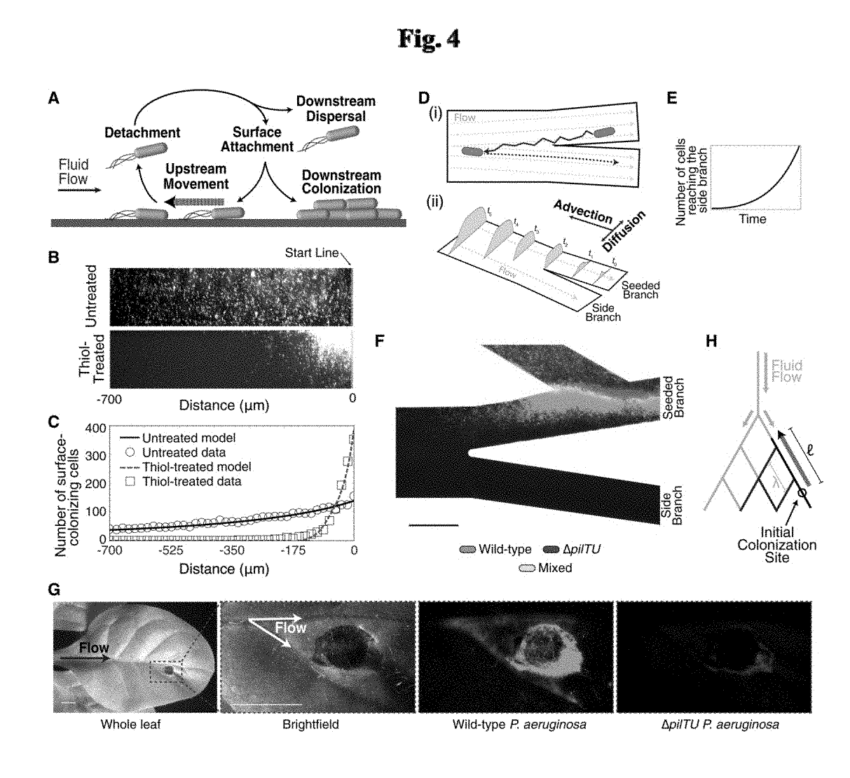

As described herein, understanding how single-cell behaviors give rise to the population-level dynamics of P. aeruginosa in flow provides a quantitative framework for how P. aeruginosa colonization is established and how infection spreads within a host. The present results indicate that P. aeruginosa responds to fluid flow as a host cue by moving in the opposite direction of the flow. By employing diversified strategies of migrating upstream, downstream, and laterally through counter-advection and diffusion, P. aeruginosa communities gain a selective growth advantage in fluid flow. This combination of behaviors enables P. aeruginosa populations to disperse rapidly throughout a fluid-filled vascular network since the population expands into a new branch each time it reaches an intersection (FIG. 4, Panel H). Since the number of cells that reach a branch junction is exponential with respect to time (FIG. 4, Panel E), the present model indicates that the upstream advancement of P. aeruginosa to a central branch node is catastrophic for the host. Indeed, the present experiments demonstrate that only a small number of cells need to reach the branch junction for the establishment of large populations downstream (FIG. 2, Panels A-B; FIG. 3, Panels A-B).

In certain non-limiting embodiments, the present model highlights strategies for minimizing colonization of flow networks. Specifically, the advantage gained by upstream dispersal can be eliminated by increasing surface adhesion. These findings have immediate implications for the design of medical devices in order to contain P. aeruginosa colonization and suggest alternative approaches to disrupting P. aeruginosa colonization and infection within hosts. The ability of bacteria to self-segregate into stably co-existing niches has significant implications for pathogens and beneficial commensals. In particular, as fluid flow is prevalent in the digestive tract, understanding how different bacterial species disperse and establish colonization in flow advances the understanding of the forces that shape the composition and structure of microbial communities, as well as the ability to manipulate these populations to improve health. Unexpected population-level dynamics emerge from the complex interactions between shear forces, the polar organization of bacterial motility structures, and the architecture of fluidic networks. For example, without wishing to be limited by any theory, the polar localization of TFPs may have evolved as an adaptation to promote competition in branched flow networks. As many bacteria possess polar-localized TFPs, upstream dispersal may be a generalized mechanism by which surface-motile bacteria colonize hosts. Thus, while flow has thus far been largely considered in the context of its effects on the motility of individual bacteria, the present study shows that flow is an important factor that drives the colonization, competition, and dispersal of bacterial populations in host environments. In certain embodiments, the upstream dispersal of P. aeruginosa provides further evidence that the bacterial response to mechanical forces plays an important role in pathogenic processes.

Compositions and Devices

The invention provides certain fluid flow networks, which are exemplified in a non-limiting manner herein. The invention should not be construed to be limited to the description herein, and contemplates any combination(s) of the embodiments recited herein.

The invention provides a fluid flow network comprising a fluid duct. In certain embodiments, the fluid duct has a first opening and a second opening. In other embodiments, fluid can flow within the fluid duct from the first opening (upstream) to the second opening (downstream). In yet other embodiments, the second opening can come into contact with a bacterium. In yet other embodiments, at least a portion of the internal surface of the fluid duct is derivatized with a coating that increases adhesion of the bacterium to the internal surface of the fluid duct as compared to the underivatized portion of the internal surface of the fluid.

In certain embodiments, at least a portion of the internal surface of the fluid duct in proximity of at least one selected from the group consisting of the first opening and the second opening is derivatized with a coating that increases adhesion of the bacterium to the internal surface of the fluid duct as compared to the underivatized portion of the internal surface of the fluid.

In certain embodiments, essentially the entire internal surface of the fluid duct is derivatized with a coating that increases adhesion of the bacterium to the internal surface of the fluid duct as compared to the underivatized internal surface of the fluid.

In certain embodiments, the bacterium comprises an upstream surface migrating bacterium. In other embodiments, the bacterium comprises Pseudomonas aeruginosa. In yet other embodiments, the bacterium comprises at least one selected from the group consisting of Neisseria gonorrhoeae, Neisseria meningitidis, Legionella pneumophila, and Streptococcus sanguinis. In yet other embodiments, the bacterium comprises at least one selected from the group consisting of Acinetobacter calcoaceticus, Aeromonas hydrophila, Azoarcus spp., Bacteroides ureolyticus, Branhamella catarrhalis, Comomonas testosterone, Dichelobacter nodosus, Eikenella corrodens, Kingella denitrificans, Kingella kingae, Legionella pneumophila, Moraxella bovis, Moraxella lacunata, Moraxella nonliquefaciens, Moraxella kingie, Mycoplasma mobile, Pseudomonas stutzeri, Pseudomonas putida, Pseudomonas syringae, Ralstonia solanacearum, Shewanella putrefaciens, Suttonella indologenes, Vibrio cholera, Wolinella spp., and/or Xylella fastidiosa

In certain embodiments, derivatization of at least a portion of the internal surface of the fluid duct in proximity of the second opening with a coating that increases adhesion of the bacterium to the internal surface of the fluid duct prevents or minimizes migration, or reduces the migration rate, of the bacterium through the fluid duct towards the first opening.

In certain embodiments, the duct is part of at least one selected from the group consisting of a catheter, intravenous line and needle.

In certain embodiments, the fluid flowing through the duct comprises a bodily fluid. In other embodiments, the bodily fluid comprises at least one selected from the group consisting of blood, serum, plasma and urine.

In certain embodiments, at least one selected from the group consisting of the first opening and second opening is in proximity to a branching point of the network, wherein the fluid duct is in fluid communication with one or more other fluid ducts.

In certain embodiments, at least a portion of the internal surface of the fluid duct in proximity to the branching point of the network is derivatized with a coating that increases adhesion of the bacterium to the internal surface of the fluid duct as compared to the underivatized portion of the internal surface of the fluid.

In certain embodiments, the coating comprises at least one chemical group selected from the group consisting of an alcohol and a thiol. In other embodiments, the coating comprises at least one protein selected from the group consisting of fibronectin, fibrin and fibrinogen. In yet other embodiments, the internal surface of the fluid duct comprises at least one selected from the group consisting of silica or glass, and wherein the coating comprises at least one silane. In yet other embodiments, the fluid flow network of the invention is part of an organism's vasculature. In yet other embodiments, the fluid duct is an implantable device that is implanted within the organism's vasculature. In yet other embodiments, the organism comprises at least one selected from the group consisting of a plant and an animal.

Methods

The invention provides certain methods of preparing derivatized fluid flow networks, which are exemplified in a non-limiting manner herein. The invention should not be construed to be limited to the description herein, and contemplates any combination(s) of the embodiments recited herein.

The invention provides a method of preventing or minimizing colonization of a fluid flow network comprising a fluid duct by a bacterium, wherein the fluid duct has a first opening and a second opening, wherein fluid can flow within the fluid duct from the first opening to the second opening, wherein the second opening comes into contact with the bacterium.

In certain embodiments, the method comprises derivatizing at least a portion of the internal surface of the fluid duct with a coating that increases adhesion of the bacterium to the internal surface of the fluid duct as compared to the underivatized portion of the internal surface of the fluid.

In certain embodiments, at least a portion of the internal surface of the fluid duct in proximity of at least one selected from the group consisting of the first opening and the second opening is derivatized with a coating that increases adhesion of the bacterium to the internal surface of the fluid duct as compared to the underivatized portion of the internal surface of the fluid.

In certain embodiments, essentially the entire internal surface of the fluid duct is derivatized with a coating that increases adhesion of the bacterium to the internal surface of the fluid duct as compared to the underivatized internal surface of the fluid.

In certain embodiments, the bacterium comprises an upstream surface migrating bacterium. In other embodiments, the bacterium comprises Pseudomonas aeruginosa. In yet other embodiments, the bacterium comprises at least one selected from the group consisting of Neisseria gonorrhoeae, Neisseria meningitidis, Legionella pneumophila, and Streptococcus sanguinis. In yet other embodiments, the bacterium comprises at least one selected from the group consisting of Acinetobacter calcoaceticus, Aeromonas hydrophila, Azoarcus spp., Bacteroides ureolyticus, Branhamella catarrhalis, Comomonas testosterone, Dichelobacter nodosus, Eikenella corrodens, Kingella denitrificans, Kingella kingae, Legionella pneumophila, Moraxella bovis, Moraxella lacunata, Moraxella nonliquefaciens, Moraxella kingie, Mycoplasma mobile, Pseudomonas stutzeri, Pseudomonas putida, Pseudomonas syringae, Ralstonia solanacearum, Shewanella putrefaciens, Suttonella indologenes, Vibrio cholera, Wolinella spp., and/or Xylella fastidiosa.

In certain embodiments, derivatizing the internal surface of the fluid duct in proximity of the second opening with a coating that increases adhesion of the bacterium to the internal surface of the fluid duct prevents or minimizes migration, or reduces the migration rate, of the bacterium through the fluid duct towards the first opening.

In certain embodiments, the duct is part of at least one selected from the group consisting of a catheter, intravenous line and needle.

In certain embodiments, the fluid flowing through the duct comprises a bodily fluid. In other embodiments, the bodily fluid comprises at least one selected from the group consisting of blood, serum, plasma and urine.

In certain embodiments, at least one selected from the group consisting of the first opening and second opening is in proximity to a branching point of the network, wherein the fluid duct is in fluid communication with one or more other fluid ducts.

In certain embodiments, at least a portion of the internal surface of the fluid duct in proximity to the branching point of the network is derivatized with a coating that increases adhesion of the bacterium to the internal surface of the fluid duct as compared to the underivatized portion of the internal surface of the fluid.

In certain embodiments, the coating comprises at least one chemical group selected from the group consisting of an alcohol and a thiol.

In certain embodiments, the coating comprises at least one protein selected from the group consisting of fibronectin, fibrin and fibrinogen.

In certain embodiments, the internal surface of the fluid duct comprises at least one selected from the group consisting of silica or glass, and wherein the coating comprises at least one silane.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. Although the description herein contains many embodiments, these should not be construed as limiting the scope of the present invention but as merely providing illustrations of some of the presently preferred embodiments of the present invention.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application. In general the terms and phrases used herein have their art-recognized meaning, which can be found by reference to standard texts, journal references and contexts known to those skilled in the art. Any preceding definitions are provided to clarify their specific use in the context of the present invention.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

Materials and Methods

General Comments:

Strains were grown in lysogeny broth to mid-exponential phase. For competition experiments, strains were grown separately and mixed in equal number immediately before loading into devices. Microfluidic devices were constructed using standard soft photolithography techniques. Effluent from each branch of the device was collected into culture tubes and incubated for 3.5 hours at 37° C. in order to amplify the total number of cells for single-cell microscopy analysis. The total cell number in the effluent was measured by optical density. P. aeruginosa colonization was tracked in Nicotiana tabacum tobacco plants that were exposed continuously to a fluorescent growth light for 7 days.

Bacterial Strains and Culture Conditions:

Strains were grown in LB Broth (Miller) (Becton, Dickinson and Company, Franklin Lakes, N.J.) at 37° C. overnight to saturation in a shaker or roller drum, back-diluted 1:100 to 1:1000, and grown at 37° C. to mid-exponential phase (an optical density at 600 nm (OD₆₀₀) corresponding to 0.1 to 0.5). For competition experiments, wild-type P. aeruginosa expressing GFP (AFS64) was mixed with an equal number of ΔpilTU P. aeruginosa expressing mCherry (AFS48), P. mirabilis (HI4320), S. aureus (RN4220), or S. Typhimurium (MET708). For experiments involving S. aureus, both P. aeruginosa and S. aureus strains were cultured in tryptic soy broth instead of LB. To test the abilities of B. subtilis and E. coli to disperse in branched networks, B. subtilis (168) or E. coli expressing YFP (MDG147) were mixed with an equal number of ΔpilTU P. aeruginosa expressing mCherry (AFS48).

Construction of an mCherry-Expressing ΔpilTU P. Aeruginosa Strain:

A strain that constitutively expresses mCherry was constructed by amplifying the hybrid sequence corresponding to the −51 to 0 region of P_{A1/04} (Lanzer, et al., 1988, Proc Natl Acad Sci U S A 85:8973-8977) and the +1 to +28 region of P_{A1/03} (Lanzer, et al., 1988, Proc Natl Acad Sci U S A 85:8973-8977) (here referred to as the P_{A1/04/03} promoter) from a PA14-derived strain that constitutively expresses GFP. The promoter sequence was joined with the sequence encoding mCherry using overlap extension, cloned into the Kpnl and HindIII sites in the plasmid pUC18-mini-Tn7T-Gm (Choi, et al., 2005, Nat. Methods 2:443-448), and integrated into the chromosome of PA14 using pTNS2 (Choi, et al., 2005, Nat. Methods 2:443-448), yielding the constitutively-expressed mCherry strain AF S27E.

The gentamicin resistance gene was flipped out from the ΔpilTU::aacC1 strain AFS19 (Shen, et al., 2012, Biophys J 103:146-151) using pFLP2, yielding a ΔpilTU::FRT strain. The P_{A1/04/03}-mCherry-aacC1 region in AFS27E was amplified by PCR and transformed into the ΔpilTU::FRT strain containing the λ Red recombination plasmid pUCP18-RedS (Lesic, et al., 2008, BMC Mol Biol 9:20), yielding the ΔpilTU::FRT mCherry strain AFS48.

Microfluidic Device Fabrication:

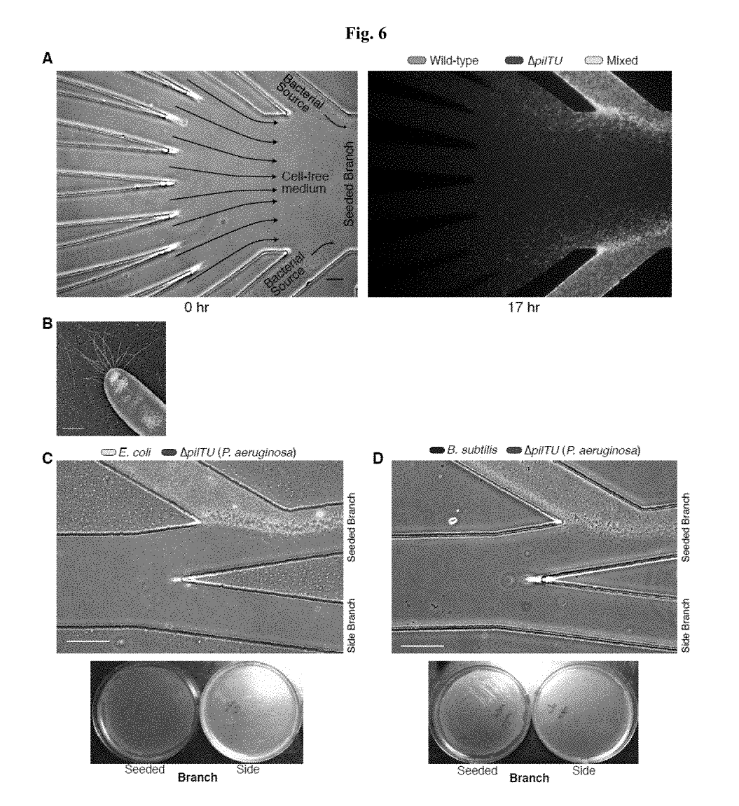

Microfluidic masks were designed using AutoCAD software (Autodesk, San Rafael, Calif.) and were printed at 20,000 dpi resolution (CAD/Art Services, Inc., Brandon, Oreg.). Molds were prepared using SU8-2000 series photoresist (MicroChem, Westborough, Mass.) and standard soft photolithography techniques. Polydimethylsiloxane (PDMS) microfluidic devices were constructed using photoresist molds and the Sylgard 184 elastomer kit (Dow Corning, Midland, Mich.) and were bonded to glass microscope slides following plasma treatment. For straight and branched flow network devices, the main channel had cross-sectional dimensions of 200 μm×50 μm (width×height). The distance between the seeded branch and the branch junction was 100 μm. In order to study the population expansion in a straight channel, this distance was increased to 800 μm. For converging branched devices (FIG. 6, Panel A), the cross-sectional dimensions of the main channel of converging branch devices was 800 μm×30 μm (width×height). The cross-section of the main channel of T-shaped devices was 100 μm×30 μm (width×height) (FIG. 3, Panel F). To increase surface adhesion, microfluidic devices were first treated with a 5:1:1 H₂O:H₂O₂:HCl solution. After 5 min at room temperature, the devices were flushed with water and air-dried. The devices were then loaded with (3-mercaptopropyl) trimethoxysilane (Sigma, St. Louis, Mo.). After 30 minutes, the devices were flushed with water to remove unreacted silane, yielding thiol-coated microfluidic devices.

Microfluidic Experiments:

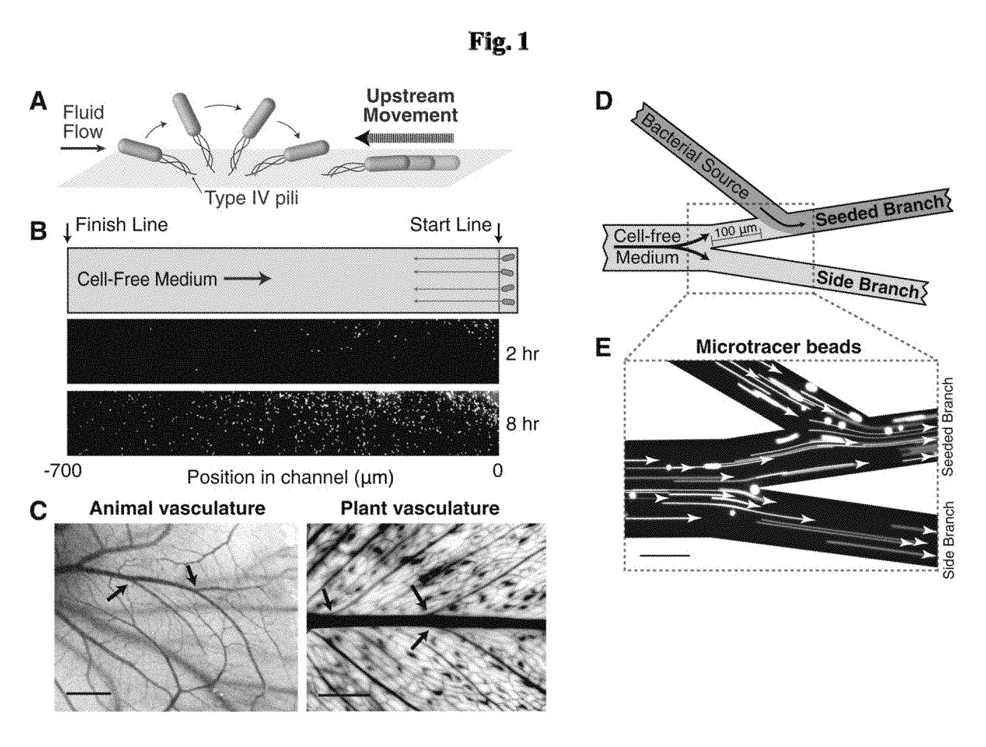

Experiments were performed at room temperature. The inlets of the microfluidic devices were connected to syringes by polyethylene tubing (BD, Franklin Lakes, N.J.) and the flow of media and cells into the devices was controlled using syringe pumps (KD Scientific, Holliston, Mass.). In order to visualize the flow pattern in branched network devices (FIG. 1, Panel E), red-fluorescent 0.50 μm diameter micro-tracer beads (Thermo Scientific, Pittsburg, Pa.) were added to the medium and imaged using fluorescence microscopy. In order to load bacterial cells into the devices, a steady flow of sterile LB was first injected at 100 μL/min into the main channels of branched microfluidic devices. Culture containing mixed bacterial species growing at mid-exponential phase were injected into the seeded branch at 1 μL/min. After approximately 15 minutes, the syringe containing bacterial culture was replaced with a syringe containing sterile LB. The flow into the main channel was steadily reduced to 1-10 μL/min while the flow to the seeded channel was kept constant. Images were acquired using phase contrast and fluorescence microscopy at 30 second to 2 minute intervals for 15 hours to track colonization in the channels. Effluent from each branch was collected into culture tubes kept at 37° C. for the duration of the experiment, which typically ran for 15 hours. The culture tubes were placed in a roller drum at 37° C. for 3.5 hours in order to amplify the total number of cells for single-cell microscopy analysis. Cultures were imaged at single-cell resolution using phase contrast and fluorescence microscopy at 100X magnification. Images were analyzed for fluorescence and cell size using our own software written in MATLAB (MathWorks, Natick, Mass.). The total number of cells in the effluent was determined by measuring the bulk OD₆₀₀ and using the estimate that an OD₆₀₀ measurement of 1 corresponds to approximately 1 x 10⁹ cells per mL of culture.

Phase Contrast and Fluorescence Microscopy:

Imaging was performed using a Nikon Ti-E microscope (Nikon, Melville, N.Y.), a 10× Plan Fluor Ph1 Nikon objective (0.3 NA) or 100× Plan Apo VC Nikon objective (1.4 NA), a Prior Lumen 200 Pro, and an Andor Clara camera (Andor Technology, South Windsor, Conn.). GFP, YFP or mCherry fluorescence was imaged using the 89002 or 89014 filter sets (Chroma, Bellows Falls, VT) with the ET490/20x excitation and the ET535/50m emission filters (for GFP), the ET500/20x excitation and the ET535/30m emission filters (for YFP), or the ET572/35x excitation and the ET632/60m emission filters (for mCherry). The trajectories of individual cells in branched network devices (FIG. 2, Panel E) were tracked by acquiring images every 30 seconds and analyzing using Nikon NIS Elements.

Leaf Vasculature Imaging:

A leaf from the plant Epipremnum aureum was cut at the node and placed in 100 MM fluorescein solution for 4 hours at room temperature. The leaf was imaged for GFP fluorescence using a fluorescence petri dish imager constructed with 472/30 nm excitation and 520/35 nm emission BrightLine single bandpass filters (Semrock, Rochester, N.Y.). Images were acquired using a Canon EOS Rebel Tli digital camera (Canon USA, Melville, N.Y.) with a Canon EF-S 18-55 mm f/3.5-5.6 IS lens.

Swimming Assay:

P. aeruginosa strains were grown overnight in LB and inoculated into freshly poured 0.3% LB agar plates using a fine tip (as described in Filloux, et al., 2014, Pseudomonas methods and protocols. In Methods in Molecular Biology, Vol. 1149. pp. 59-65), grown for 15 hours at 37° C., and imaged using a Syngene gel doc system (Syngene, Frederick, Md.).

Planktonic Growth Rates and Competitions:

Strains were grown overnight in LB broth at 37° C. with shaking, back-diluted 1:1000 into LB, and measured for optical density at 600 nm (0D₆₀₀) between the values of 0.05 and 0.6. The growth rates were determined by fitting the OD₆₀₀ curves with an equation for exponential growth. For competitions, fluorescent protein-expressing or unlabeled strains were cultured overnight in LB broth at 37° C., back-diluted 1:1000, grown to OD₆₀₀=0.1 and mixed in equal volumes. The co-cultures were grown for 3.5 hours and imaged using phase contrast and fluorescence microscopy at 100× magnification. The relative abundance of each strain was determined by analyzing images for fluorescence in individual cells using our own software written in MATLAB (MathWorks, Natick, Mass.).

Plant Colonization:

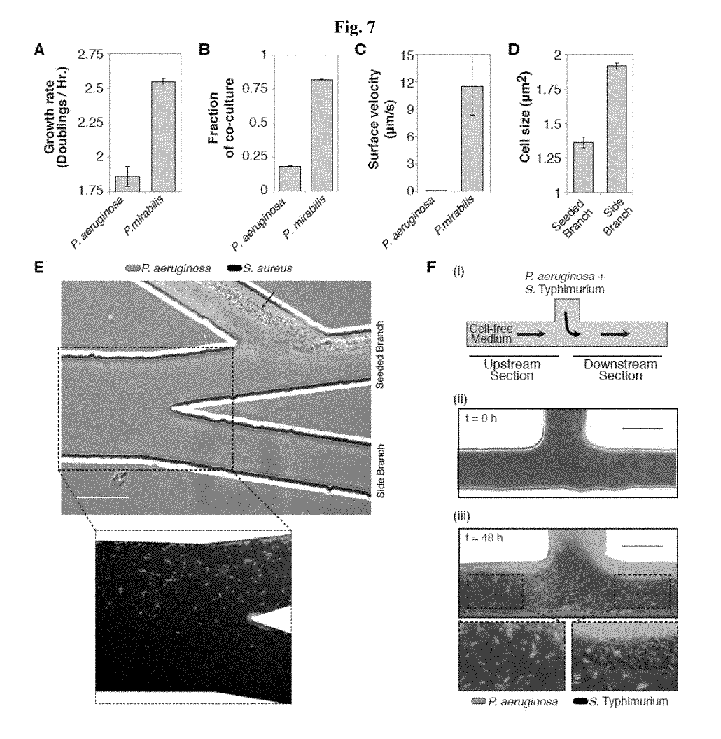

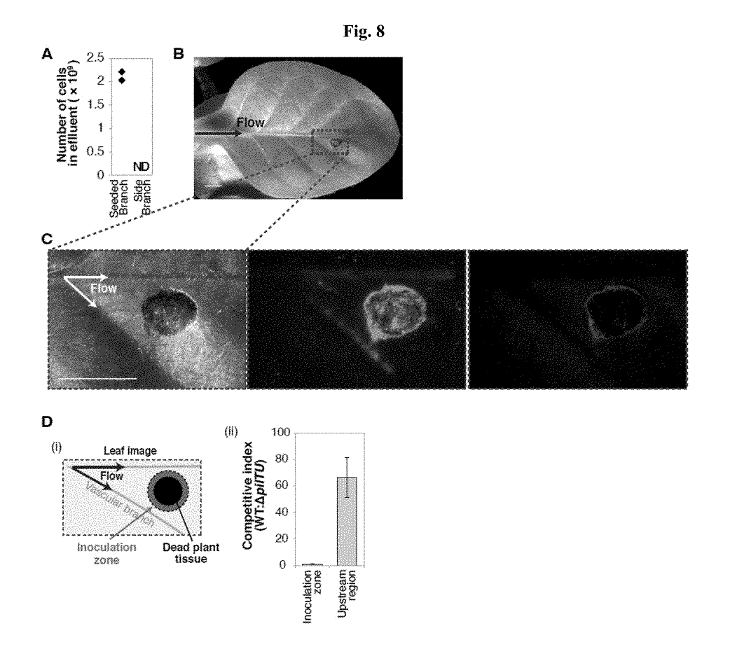

Nicotiana tabacum tobacco plants were cultivated from seeds and grown in a greenhouse until leaves were approximately 6 cm in length. An equal mixture of wild-type P. aeruginosa expressing GFP (AFS64) or ΔpilTUcells expressing mCherry (AFS48) were inoculated from LB plates, grown overnight at 37° C., diluted 1:100, grown until mid-exponential phase, mixed in equal numbers, and inoculated into plant leaves using syringe infiltration. Plants were exposed continuously to a fluorescent growth light (Ottlite Technology, Tampa, Fla.) for 7 days, and imaged using a Leica M205 FA microscope (Leica Microsystems, Buffalo Grove, Ill.) with Leica GFP3 and mCherry filter sets and Leica LAS AF imaging software. The fluorescence intensities from GFP and mCherry images were quantified using ImageJ 1.440 Bethesda, Md.). Masks were created that outlined the inoculation zone and the encompassing area that contained nearby vascular branches (FIG. 8, Panel D, (i)). The fluorescence in the upstream region was computed by subtracting the fluorescence intensity in the encompassing area by that of the inoculation zone. The competitive indexes for the inoculation zone and the upstream region were computed by dividing the GFP fluorescence intensities by those of mCherry.

Transmission Electron Microscopy:

Wild-type P. aeruginosa cells were grown overnight in 1% (w/v) tryptone broth to saturation, diluted 1:100, grown to mid-exponential phase, placed on 200 mesh carbon film glow-discharged grids, and stained with 1% uranyl acetate. Type IV pili were visualized on a Zeiss912AB Transmission Electron Microscope equipped with an Omega Energy Filter using an electron beam at 80 kV and at a magnification of 4000×. Images were captured using an AMT digital camera (Advanced Microscopy Techniques, Woburn, Mass.).

Surface Motility Velocities:

The velocities of P. aeruginosa and P. mirabilis cells on surfaces in the absence of flow were measured by picking cells from petri dishes, stabbing through 0.75% agarose pads (made using LB) on cover-glass bottom dishes (World Prescision Instruments, Sarasota, Fla.), and imaging the cover-glass surface. P. aeruginosa and P. mirabilis were imaged at 5 minute or 40 millisecond intervals, respectively. Cells were tracked using Nikon NIS Elements (Nikon, Melville, N.Y.) software. The velocity of P. aeruginosa was measured for clusters of twitching cells and the velocity of P. mirabilis was measured for individual cells.

Non-limiting experimental examples relating to the present invention follow.

### Example 1: Upstream Dispersal Involves Multiple Phenotypically Diverse Single-Cell Motility Modes

To investigate P. aeruginosa colonization dynamics in flow, the leading edge of a population was first imaged in a linear microfluidic channel. Cell-free medium was flowed steadily through the device (200×50 μm, width 3 height) at wall shear stresses between 0.2 to 2 Pa (1-10 μl/min, υ_{fluid}=2-20 mm/s), which correspond to the dimensions, shear stresses, and flow speeds typically observed in the vasculature of plants and animals. These flow rates are significantly higher than typical bacterial swimming rates. Cells were initially seeded at one side of the channel behind a “start line” (FIG. 1, Panel B). In the presence of flow, cells exhibited three distinct motility behaviors: (1) movement in the opposite direction of the flow toward the “finish line” (700 μm upstream), (2) detachment from the surface (and subsequent downstream movement with the flow), or (3) no motility. After 2 hours, the fastest cells at the leading edge reached the upstream finish line (FIG. 1, Panel B), whereas slower cells continued upstream migration. Cells were also found at the start line and downstream. The dispersal of the population to upstream and downstream regions in the device (collectively referred to as “upstream dispersal”) is due to the phenotypic diversity of motility modes within a single population. Without wishing to be limited by any theory, as the behavioral heterogeneity at the single-cell level benefits the community as a whole, this could represent a form of bet hedging by P. aeruginosa that enables the species to explore upstream and downstream environments while maintaining colonization of the initial environment.

### Example 2: P. aeruginosa Gains a Selective Growth Advantage by Dispersing Upstream

It was then investigated whether upstream dispersal provides bacteria with a selective growth advantage over bacteria that lack this motility mode. For these studies, the microfluidic device was modified to incorporate a branched network geometry, which enabled one to quantify colonization, competition, and dispersal in the same device. Branched networks are a defining feature of animal and plant vasculatures (FIG. 1, Panel C), with bifurcation of vasculature promoting the uniform distribution of nutrients. To establish the flow pattern in this complex network (FIG. 1, Panel D), micro-tracer beads were imaged to verify that the flow was laminar, stable, and unidirectional (FIG. 1, Panel E).

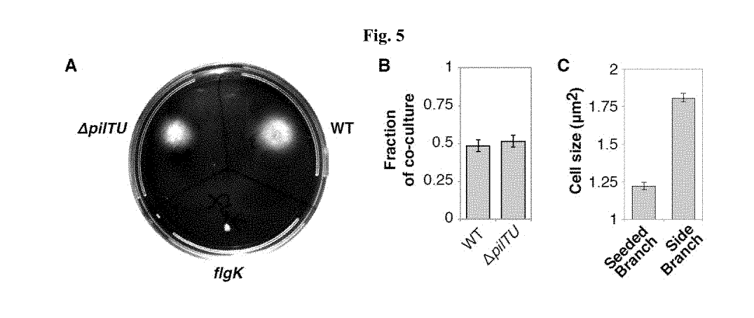

One branch, the “seeded branch” (FIG. 1, Panel D), was inoculated with an equal number of wild-type P. aeruginosa (expressing GFP) and ΔpilTU mutants (expressing mCherry) that are defective in surface motility because they lack the TFP PilT and PilU retraction motors but retain swimming motility (FIG. 5, Panel A). After 15 hours, wild-type cells colonized both the seeded and side branches, whereas the ΔpilTU cells colonized only the seeded branch (FIG. 2, Panel A). The fluid effluent from each branch, which contains both planktonic (swimming) cells and cells that detached from the surface, was collected and cultured for 3.5 hours at 37° C. in order to characterize cell physiology and determine the relative proportion of wild-type and ΔpilTU cells using single-cell fluorescence microscopy. The effluent from the seeded branch contained about 2.4×10⁹ cells, of which 60% were wild-type and 40% were ΔpilTU cells (FIG. 2, Panels B-D). In contrast, the side branch contained only wild-type cells (FIG. 2, Panels A-D). The total number of wild-type cells in both branches outnumbered ΔpilTU cells by more than 2-fold (about 1.3×10-more cells), indicating that upstream migration results in a selective growth advantage. The greater number of wild-type cells is not due to a difference in growth rates as both strains are represented comparably in planktonic co-culture competitions (FIG. 5, Panel B). Furthermore, wild-type cells from the side branch were 40% larger than those from the seeded branch (FIG. 2, Panel C; FIG. 5, Panel C), indicating the increased availability of nutrients in the side branch. Thus, by entering the side branch, the upstream-migrating population escapes nutrient limitation imposed by the competing ΔpilTU cells.

### Example 3: Dispersal through Zigzag Paths on Surfaces

Given that cells that move upstream should simply return downstream along the same streamline when they are released from the surface, the present findings raise the question of how P. aeruginosa dispersed to the side branches. By tracking the movements of individual cells (FIG. 2, Panel E), upstream motility was shown to have a component whose direction is perpendicular to the flow. P. aeruginosa cells always migrated in a zigzag path, with some trajectories crossing over into streamlines that flow into the side branch. Cells that entered these streamlines were frequently detached from the surface, carried into the side branch, and subsequently re-attached to the surface of the side branch. This zigzag motion was observed in upstream movement in linear channels and in a network geometry in which branches converge at the intersection instead of diverging (FIG. 6, Panel A). Without wishing to be limited by any theory, the motion can be attributed to the radial organization of TFPs at the cell pole (FIG. 6, Panel B) and their non-synchronous activity. Zigzag trajectories are also observed through flagellar-mediated swimming in sperm rheotaxis, indicating that similar upstream motions can be achieved using distinct motility mechanisms.

An alternative explanation for the presence of bacteria in the side channels is through flagella-mediated upstream movement. However, ΔpilTU mutants retain swimming motility (FIG. 5, Panel A) but do not reach the side channel (FIG. 2, Panels A-D), supporting the non-limiting conclusion that migration to the side channel is driven by surface motility. It was also tested whether B. subtilis or E. coli migrates toward the side channel, as both move upstream through flagella-mediated rheotactic motion. After 15 hours of continuous flow in which either species was inoculated into the seeded channel, no cells were found on the surface or in effluent from the side channel (FIG. 6, Panels C-D), suggesting that the flagella-mediated rheotactic behavior does not result in dispersal. Together, the present data show that colonization and dispersal is due to surface-mediated upstream migration.

### Example 4: Selective Advantage Enables Competition against Faster-Growing Pathogens

In natural settings, bacteria compete against other species for shared resources. To determine whether upstream dispersal confers P. aeruginosa with a competitive advantage against other species, the seeded branch was co-inoculated with equal numbers of P. aeruginosa and Proteus mirabilis, a Gram-negative bacterium that grows at a faster rate than P. aeruginosa (FIG. 7, Panels A-B) and moves on surfaces at significantly higher velocities (FIG. 7, Panel C). Both pathogens cause nosocomial infections, colonize the urinary tract and gut, and may be considered natural competitors. In addition, P. mirabilis swims using a flagella-mediated mechanism similar to that of E. coli, which suggests that P. mirabilis can move upstream.

Media was flowed continuously for 15 hours (FIG. 3, Panel A), effluent containing planktonic and surface-detached cells from each branch was collected, and the analysis described elsewhere herein was performed (FIG. 3, Panels B-D). P. mirabilis outgrew P. aeruginosa in the seeded branch and accounted for 85% of the bacteria in the branch effluent (FIG. 3, Panel D). In contrast, only P. aeruginosa cells colonized the side branch (FIG. 3, Panels A and C-D). Examination of individual cell trajectories showed that P. mirabilis cells did not disperse upstream, thereby explaining their restriction to the seeded branch. Furthermore, P. aeruginosa cells in the seeded branch were 29% smaller than those in the side branch (FIG. 3, Panels C-D), indicating that P. aeruginosa cells were nutrient limited in the co-culture with P. mirabilis. These results demonstrate that upstream migration by a surface-motility mechanism enables P. aeruginosa to self-segregate from the co-culture to colonize a separate niche in flow. P. aeruginosa thus gains a competitive advantage over a bacterium that would otherwise outgrow P. aeruginosa in the absence of flow. Similar findings were observed when P. aeruginosa was co-cultured with Staphylococcus aureus (FIG. 7, Panel E), which co-colonizes the lungs of cystic fibrosis patients alongside P. aeruginosa, and with the pathogen Salmonella enterica serovar Typhimurium (FIG. 7, Panel F). These results demonstrate that in a flow environment, bacterial species can self-organize to co-exist in separate micro-environments, despite one species having an apparent growth advantage over the other.

### Example 5: Upstream Dispersal Is Characterized by Counter-advection and Lateral Diffusion

To improve the ability to understand and ultimately disrupt P. aeruginosa dispersal and colonization, a quantitative model of upstream dispersal was developed. Upstream migration within a population is a phenotypically diverse cycle consisting of upstream movement on surfaces, detachment from the surface, and downstream re-attachment to the surface (FIG. 4, Panel A). As cells move upstream along the flow axis (x axis), cells also move and repeatedly switch direction along the transverse (y) axis, resulting in a zigzag path (FIG. 2, Panel E). This behavior in single cells is described by the differential equation

$$\frac{\partial σ ( x , y , t )}{\partial t} = ( α - β ) σ ( x , y , t ) - v \frac{\partial σ ( x , y , t )}{\partial x} + D \frac{{\partial }^{2} σ ( x , y , t )}{\partial {y}^{2}} ,$$

where σ(x,y,t) is the population density of cells at positions x and y and at time t that travel upstream at a velocity υ, duplicate at a rate α, and detach from the surface at a rate β; D is the effective diffusion coefficient. Upstream dispersal is thus a novel type of movement that is counter-advective along the flow axis and diffusive along the lateral axis. This equation was solved for a population of cells with distinct upstream velocities in a branched flow network. The model accurately predicts the population density distribution data in linear channels (FIG. 4, Panels B-C) and in branched flow networks and shows that the number of cells that enter the side branch (FIG. 4, Panel D) increases exponentially with time (FIG. 4, Panel E). As bacterial growth is also exponential with time, the combined effects of exponential influx and growth result in rapid colonization of the side branch.

A prediction of the model that decreasing the surface detachment rate (B) and migration velocity (υ) restricts population expansion and prevents dispersal to the side branch was tested. Channel surfaces were coated with the thiol compound (3-mercaptopropyl) trimethoxysilane to promote sulfide bond formation with proteins on the bacterial cell surface, thereby increasing cell adhesion to the surface (i.e., making the surface more “sticky”). Thiol-treated linear channels inhibited the expansion of the P. aeruginosa population in linear channels (FIG. 4, Panels B-C) and prevented cells from entering the side branch (FIG. 4, Panel F; FIG. 5, Panel A), in good agreement with the predictions of the model of upstream dispersal.

### Example 6: Upstream Dispersal Promotes Colonization in Plant Vasculature

To establish the role of upstream dispersal in the colonization of the vasculature of a natural host of P. aeruginosa, colonization of the tobacco plant Nicotiana tabacum was followed. In these plants, fluid and nutrients flow from the main stem toward the periphery of the leaf vasculature in a branched network (FIG. 4, Panel G). Plant leaves were inoculated with an equal mixture of wild-type P. aeruginosa (expressing GFP) and ΔpilTU cells (expressing mCherry) at the periphery using syringe infiltration. After 7 days, wild-type cells were found in the vasculature upstream from the inoculation site (toward the main stem) (FIG. 4, Panel G; FIG. 8, Panels B-D). In contrast, ΔpilTU cells were found only at the inoculation site, resulting in a much more localized infection. The results are thus consistent with upstream motility representing an important factor driving branched vasculature colonization in vivo.

### Example 7: Model of Bacterial Dispersal by Upstream Migration

Motion along the Flow Axis (x-axis)

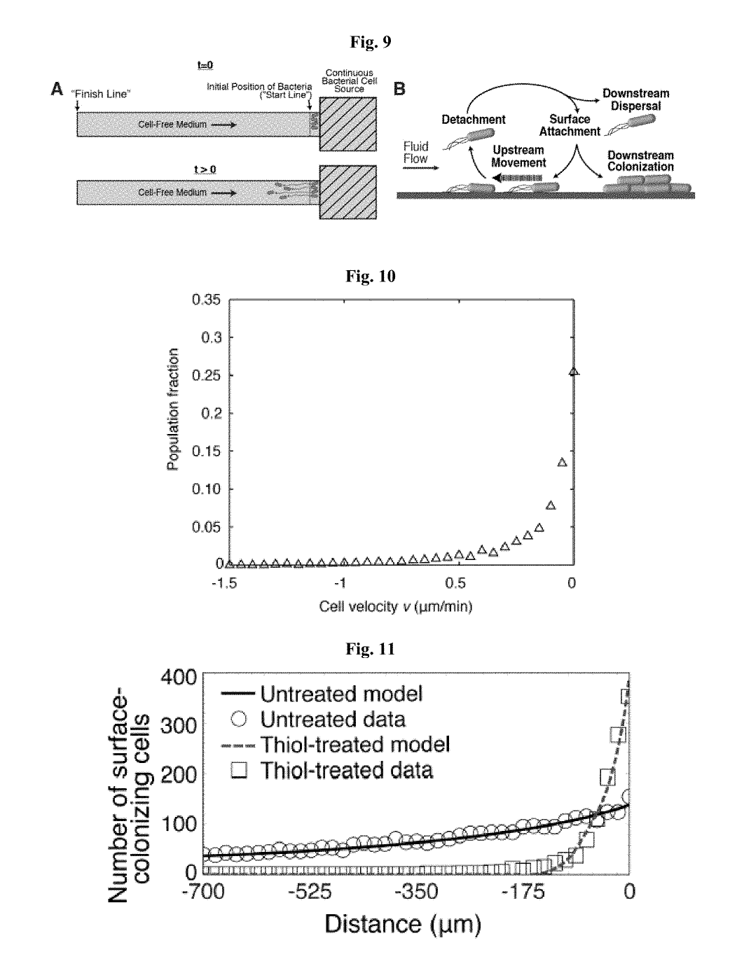

An equation that describes the dynamics of a cell population as it migrates upstream in a linear channel was derived, as depicted in FIG. 1, Panel B, and FIG. 9. Cell-free medium flows from left to right in the schematic towards the bacterial cell source. Cells are continuously seeded behind the “start line” from the source for the entire experiment. At t>0, cells move from the start line towards the finish line (in the opposite direction of the flow).

The number of cells at a fixed position x was first considered. Increasing x corresponds to the downstream direction and υ>0 corresponds to movement towards the downstream direction. Conversely, decreasing x corresponds to the upstream direction and υ<0 corresponds to movement towards the upstream direction. The change in the number of cells during the time interval Δt and along the spatial interval Δx is increased by duplication of the cells at a rate a and is decreased by detachment from the surface at a rate B. Cells traveling at a velocity υ>0 enter from an upstream region and leave at a velocity υ>0 to a downstream region. In this analysis, the contribution of re-attached cells was ignored. An equation for σ, the number density (number per unit length) of cells, was constructed as:

(σ_{x,t+Δt}−σ_{x,t})Δx=(αΔt)σ_{x,t}Δx−(βΔt)σ_{x,t}Δx+σ_{x-Δxt}(υΔt)−σ_{x,t}(υΔt). (1)

Dividing both sides by ΔxΔt and taking the limits Δx→0 and Δt→0, a differential equation that has a form that is similar to the advection equation is obtained:

$$\frac{\partial σ ( x , t )}{\partial t} = ( α - β ) σ ( x , t ) - v \frac{\partial σ ( x , t )}{\partial x} . \qquad ( 2 )$$

This equation has the solution:

σ(x,t)=e^{(α-β)t}f(x−υt). (3)

where f is an arbitrary function. Since the value off is unchanged as one moves along the x-axis a distance υt, the solution illustrates a wave that travels at velocity v. The amplitude of the waves grows exponentially with time for (α−β)>0 or decays exponentially with time for (α−β)<0.

In order to understand the movement of a cell population, the movement of a single cell was first considered. A single cell was represented by the Dirac delta function and substituted for the arbitrary function fin Equation (3):

σ_{single cell}(x,t)=e^{(α-β)t}δ(x−υt). (4)

This solution describes a singularity that moves along the x-axis with velocity υ. As the delta function is infinite at x=υt and zero everywhere else, σ_{single cell} does not have a physical interpretation. However, by integrating σ_{single cell} along the x-axis, the total number of cells is obtained, which is equal to the growth/decay term e^{(α-β)t} multiplied by unity:

Total number of cells(t)=∫_{-∞}^{∞}σ_{single cell}(x, t)dx=e^{(α-β)t}∫_{-∞}^{∞}δ(x−υt)dx=e^{(α-β)}t. (5)

The function σ_{single cell} thus describes the number density for an exponentially growing or decaying localized pulse that moves with velocity v along the x-axis.

In a population of P. aeruginosa cells, sub-populations move upstream at different velocities. To describe the movement of an entire cell population, one must therefore account for each sub-population. A single sub-population that has a cells at t=0 and that moves coherently with velocity υ is described by:

σ_{sub-population}(x,t)=ae^{(α-β)t}δ(x−υt). (6)

To describe an entire population, the solutions of individual sub-populations were superimposed:

$${σ}_{population} ( x , t ) = {e}^{( α - β ) t} {\sum }_{i = 1}^{n} {α}_{i} δ ( x - {v}_{i} t ) , \qquad ( 7 )$$

where aᵢ is the number of cells in each sub-population that moves with a velocity υᵢ.

The distribution of velocities in a population can be described by a continuous normalized velocity probability density function p(υ), which gives the relative fraction of cell sub-populations moving at velocity υ. The sub-population coefficient aᵢ was replaced in Equation (7) with p(υ) and a normalization factor A, the summation was replaced by an integral, and integration was done over all upstream velocities (−∞<υ<0):

$$\begin{matrix}{σ}_{population} ( x , t ) = {( A e )}^{( α - β ) t} {\int }_{- ∞}^{0} p ( v ) δ ( x - vt ) dv & ( 8 ) \\ = A \frac{{e}^{( α - β ) t}}{t} p ( x / t ) . & ( 9 )\end{matrix}$$

Equation (9) is a general solution that describes the population density of a single population that travels upstream with velocity probability density function p from the start line (FIG. 9). In the experiments, additional populations are continuously seeded behind the start line. To account for this, integration was performed over the lifetime of the experiment:

$${σ}_{experiment} ( x , t ) = A {\int }_{0}^{k} \frac{{e}^{( α - β ) δ}}{δ} p ( x / s ) ds . \qquad ( 10 )$$

The flux density (the number of cells that move past x per unit length per unit time) can be found by taking the time-derivative

$$\frac{\partial }{\partial t} {σ}_{experiment} ,$$

which is simply Equation (9).

Comparison with Experimental Data

In order to compare experimental data from the device in FIG. 9 with the present model, the velocity distribution in experimental data (FIG. 10) was used to approximate the velocity probability density function (pdf) p in Equation (10).

Factors such as wall shear stress can affect the average population velocity. As pdfs are not available for all average population velocities, a dimensionless scaling parameter μ₀ for μ₀>0 was introduced into the velocity pdf by making the substitution v→μ₀ υ. As shown below, this parameter inversely scales the average population velocity. The normalization of p is considered with the substitution:

∫_{-∞}⁰p(υ)dυ=∫_{-∞}⁰(μ₀υ)μ₀dυ=1 (11)

Thus, the form of the normalized pdf with the substitution is:

p(υ)=μ₀p(μ₀υ). (12)

To understand how μ₀ affects the average population velocity, the unmodified population average velocity was first computed and the substitution υ→μ₀υ was performed:

<υ>=∫_{-∞}⁰υp(υ)dυ=∫_{-∞}⁰(μ₀υ)p(μ₀υ)μ₀dυ. (13)

The parameter μ₀ thus inversely scales the average population velocity:

$$〈 v 〉 {μ}_{0} = {\int }_{- ∞}^{0} {v μ}_{0} p ( {μ}_{0} v ) dv = \frac{1}{{μ}_{0}} 〈 v 〉 . \qquad ( 14 )$$

μ₀ was incorporated into σₚₒₚᵤₗₐₜᵢₒn (Equation (8)), arriving at equations that describe the population density for a single population and the continuously seeded populations in the experiments:

$$\begin{matrix}{σ}_{population} ( x , t ) = {μ}_{0} A \frac{{e}^{( α - β ) t}}{t} p ( {μ}_{0} \frac{x}{t} ) & ( 15 ) \\ {σ}_{experiment} ( x , t ) = {μ}_{0} A {\int }_{0}^{t} \frac{{e}^{( α - β ) s}}{s} p ( {μ}_{0} \frac{x}{s} ) ds . & ( 16 )\end{matrix}$$

The effect of varying μ₀ on the population density was considered. Increasing μ₀ (lowering the average velocity) has the effect of compressing the pdf (FIG. 10) so that all velocities are smaller on average. In the cell population, this corresponds to a condition in which all cells in the population move slower. Thus, increasing μ₀ should slow the population advance so that the population remains closer to the “start line.”

Equation (16) also has the fitting parameters a and B. As the growth rate is expected to be fixed in the experiments, α is interpreted as a constant. In addition, as the number of cells that are seeded behind the start line does not vary, the scaling constant A is interpreted to be fixed in the experiments. Thus, the model has two free parameters: the average population velocity scaling parameter (μ₀) and the surface detachment rate (B). Equation (16) was plotted for different values of μ₀ and β and show results for two value pairs (FIG. 11, same as FIG. 4, Panel C). Good correspondence was observed between the experimental data and the model.

Population Expansion in a Branched Flow Network

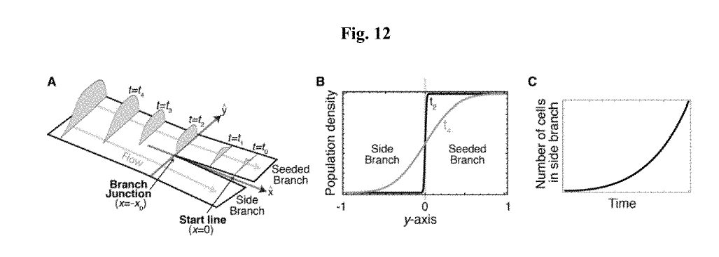

Here, the dynamics of population expansion in a branched flow network is described (FIG. 12). Bacterial cells are initially loaded into one branch (seeded branch) and travel upstream to the branch junction where the main channel splits into two separate branches. As cells move up the seeded branch along the x-axis, they diffuse along the transverse axis (y-axis). At the branch junction, cells are uniformly distributed along they-axis in the seeded branch and there are no cells in the side branch. The population density of zero in the side branch and a constant density in the seeded branch resembles a step function along the y-axis (FIG. 12, Panel 12).

As the population continues upstream past the junction along the x-axis, it diffuses along the y-axis into the side branch streamlines (FIG. 12, Panel A). To find a solution for motion along the y-axis, a solution is found to the diffusion equation in y:

$$\frac{\partial σ ( y , t )}{\partial t} = D \frac{{\partial }^{2} σ ( y , t )}{\partial {y}^{2}} , \qquad ( 17 )$$

where D is the effective diffusion coefficient. At t=0, the function should resemble a step function:

$$σ ( y , 0 ) = \{ \begin{matrix}0 & for y < 0 \\ 1 & for y \geq 0\end{matrix} . \qquad ( 18 )$$

A solution to Equation (17) that satisfies this initial condition is:

$$σ ( y , t ) = \frac{1}{2} + \frac{1}{\sqrt{}} {\int }_{0}^{\frac{y}{\sqrt{4 Dt}}} {e}^{- {s}^{2}} ds . \qquad ( 19 )$$

In particular, in the limit t→0, a step function is recovered:

$${lim}_{t \to 0} σ ( y , t ) = \{ \begin{matrix}0 & for y < 0 \\ 1 & for y \geq 0\end{matrix} . \qquad ( 20 )$$

Furthermore, it is required that the cells remain bounded within the channel (−1≦y≦1) for lifetime of the experiment. To check for this, Equation (19) is rewritten in the form of the error function erf(z), which is defined as

$$erf ( z ) = \frac{2}{\sqrt{}} {\int }_{0}^{z} {e}^{- {s}^{2}} ds$$

and integrate over y, and thus:

$${\int }_{- 1}^{1} σ ( y , t ) dy = 1 + \frac{1}{2} {\int }_{- 1}^{1} erf ( \frac{y}{\sqrt{4 Dt}} ) dy . \qquad ( 21 )$$

As the erf is an odd function, the second term is zero and the integration is thus independent of time. Equation (19) thus gives the population density (number per unit length) in the branched channel along the y-axis.

In order to compute the number of cells that move into the streamlines of the side branch (−1≦y≦0), the cell population in FIG. 12, Panel A that reaches the junction at t=t₂ is considered. The population profile along the y-axis is given by multiplying the number of cells at the branch junction (Equation (16)) at t=t₂ and x=−x₀ by σ (Equation (19)) at a time t>t₂:

σₑₓₚₑᵣᵢₘₑₙₜ(−x₀, t₂)σ(y, t−t₂). (22)

To find the total number of cells that cross over into the streamlines destined for the side branch, integration is implemented over the lifetime of the experiment and over the region along the y-axis corresponding to the side branch (−1≦y≦0):

Number of cells side branch(t)=∫₋₁⁰∫₀^{t}σₑₓₚₑᵣᵢₘₑₙₜ(−x₀, s)σ(y, t−s) ds, dy, (23)

where t≧s. The number of cells that cross into the side branch streamline region resembles a function that increases exponentially with time (FIG. 12, Panel C).

Equations for Bacterial Upstream Dispersal Inflow

Here, the equations that describe upstream dispersal in flow networks are summarized. The two-dimensional differential equation for cells moving upstream is found by incorporating the diffusive term from Equation (17) into Equation (2), arriving at the equation:

$$\frac{\partial σ ( x , y , t )}{\partial t} = ( α - β ) σ ( x , y , t ) - v \frac{\partial σ ( x , y , t )}{\partial x} + D \frac{{\partial }^{2} σ ( x , y , t )}{\partial {y}^{2}} , \qquad ( 24 )$$

where D is the effective diffusion coefficient that gives the rate of lateral diffusion. By combining the solutions for each dimension (Equations (3) and (19)), a solution for Equation (24) was found:

$${σ}_{single cell} ( x , y , t ) = {e}^{{( α - β )}^{t}} f ( x - vt ) ( \frac{1}{2} + \frac{1}{\sqrt{}} {\int }_{0}^{\frac{y}{\sqrt{4 Dt}}} {e}^{- {s}^{2}} ds ) . \qquad ( 25 )$$

For a single population of cells that can be described by a velocity probability density function p(μ₀υ), the analysis that yielded Equation (15) was repeated, arriving at:

$${σ}_{population} ( x , y , t ) = {μ}_{0} A \frac{{e}^{( α - β ) t}}{t} p ( {μ}_{0} \frac{x}{t} ) ( \frac{1}{2} + \frac{1}{\sqrt{}} {\int }_{0}^{\frac{y}{\sqrt{4 Dt}}} {e}^{- {s}^{2}} ds ) . \qquad ( 26 )$$

where A is a constant equal to the number of cells in the population as t→0, μ₀ inversely scales the average population velocity, α is the growth rate, and β is the detachment rate. Equation (26) thus describes the population density (number per unit area) of bacterial cells that move upstream through counter-advection while diffusing laterally through a branched flow network.

TABLE 1

| | | |
|---|---|---|
| Strain | Description/Genotype | Reference/Source |

Pseudomonas aeruginosa

| | | |
|---|---|---|
| PA14 (Wildtype) | Human clinical isolate | Rahme, et al., 1995, Science |
| | | 268: 1899-1902 |
| AFS19(ApilTU) | PA14 ΔpilTU::aacC1 | Shen, et al., 2012, Biophys J |
| | | 103: 146-151 |
| AFS27E (mCherry) | PA14 attTn7::[P_{A1/04/03}-mCherry] | Present study |
| AFS48 (mCherry | PA14 attTn7::[P_{A1/04/03}-mCherry] | Present study |
| ΔpilTU) | ΔpilTU::FRT | |
| AFS64 (GFP) | PA14 attTn7::[P_{A1/04/03}-gfp] | Siryaporn, et al., 2014, PNAS USA |
| | | 111, 16860-16865 |
| pilT | PA14 pilT::Mar2xT7 | Liberati, et al., 2006, PNAS USA |
| | | 103: 2833-2838 |
| flgK | PA14 flgK::Tn5 | O'Toole, et al., 1998, Mol. |
| | | Microbiol. 30: 295-304 |

Bacillus subtilis

| | | |
|---|---|---|
| 168 | trpC2 | Spizizen, 1958, PNAS USA |
| | | 44: 1072-1078. |

Escherichia coli

| | | |
|---|---|---|
| MDG147 | MG1655 Φ(ompC-cfp) Φ(ompF-yfp) | Batchelor, et al., 2004, J Bacteriol |
| | | 186: 7618-7625 |

Proteus mirabilis

| | | |
|---|---|---|
| HI4320 (Wildtype) | Human clinical isolate | Pearson, et al., 2008, J Bacteriol |
| | | 190: 4027-4037; Warren, et al., |
| | | 1982, J. Infect. Dis. 146: 719-723 |

Staphylococcus aureus

| | | |
|---|---|---|
| RN4220 | Derived from clinical isolate | Kreiswirth, et al., 1983, Nature |
| | NCTC8325 | 305: 709-712 |

Salmonella enterica
serovar Typhimurium

| | | |
|---|---|---|
| MET708 | rpsL, derived from chicken tissue | Taga, et al., 2003, Mol Microbiol |
| | isolate | 50: 1411-1427 |

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the present invention. The appended claims are intended to be construed to include all such embodiments and equivalent variations.

What is claimed is:

1. A fluid flow network comprising a fluid duct, wherein the fluid duct has a first opening and a second opening, wherein fluid can flow within the fluid duct from the first opening (upstream) to the second opening (downstream), wherein the second opening can come into contact with a bacterium, wherein at least a portion of the internal surface of the fluid duct is derivatized with a coating that increases adhesion of the bacterium to the internal surface of the fluid duct as compared to the underivatized portion of the internal surface of the fluid.

2. The fluid flow network of claim 1, wherein at least a portion of the internal surface of the fluid duct in proximity of at least one selected from the group consisting of the first opening and the second opening is derivatized with a coating that increases adhesion of the bacterium to the internal surface of the fluid duct as compared to the underivatized portion of the internal surface of the fluid.

3. The fluid flow network of claim 1, wherein essentially the entire internal surface of the fluid duct is derivatized with a coating that increases adhesion of the bacterium to the internal surface of the fluid duct as compared to the underivatized internal surface of the fluid.

4. The fluid flow network of claim 1, wherein the bacterium comprises an upstream surface migrating bacterium.

5. The fluid flow network of claim 4, wherein the bacterium comprises Pseudomonas aeruginosa.

6. The fluid flow network of claim 4, wherein the bacterium comprises at least one selected from the group consisting of Neisseria gonorrhoeae, Neisseria meningitidis, Legionella pneumophila, and Streptococcus sanguinis.

7. The fluid flow network of claim 4, wherein the bacterium comprises at least one selected from the group consisting of Acinetobacter calcoaceticus, Aeromonas hydrophila, Azoarcus spp., Bacteroides ureolyticus, Branhamella catarrhalis, Comomonas testosterone, Dichelobacter nodosus, Eikenella corrodens, Kingella denitrificans, Kingella kingae, Legionella pneumophila, Moraxella bovis, Moraxella lacunata, Moraxella nonliquefaciens, Moraxella kingie, Mycoplasma mobile, Pseudomonas stutzeri, Pseudomonas putida, Pseudomonas syringae, Ralstonia solanacearum, Shewanella putrefaciens, Suttonella indologenes, Vibrio cholera, Wolinella spp., and/or Xylella fastidiosa.

8. The fluid flow network of claim 1, wherein derivatization of at least a portion of the internal surface of the fluid duct in proximity of the second opening with a coating that increases adhesion of the bacterium to the internal surface of the fluid duct prevents or minimizes migration, or reduces the migration rate, of the bacterium through the fluid duct towards the first opening.

9. The fluid flow network of claim 1, wherein the duct is part of at least one selected from the group consisting of a catheter, intravenous line and needle.

10. The fluid flow network of claim 1, wherein the fluid flowing through the duct comprises a bodily fluid.

11. The fluid flow network of claim 10, wherein the bodily fluid comprises at least one selected from the group consisting of blood, serum, plasma and urine.

12. The fluid flow network of claim 1, wherein at least one selected from the group consisting of the first opening and second opening is in proximity to a branching point of the network, wherein the fluid duct is in fluid communication with one or more other fluid ducts.

13. The fluid flow network of claim 12, wherein at least a portion of the internal surface of the fluid duct in proximity to the branching point of the network is derivatized with a coating that increases adhesion of the bacterium to the internal surface of the fluid duct as compared to the underivatized portion of the internal surface of the fluid.

14. The fluid flow network of claim 1, wherein the coating comprises at least one chemical group selected from the group consisting of an alcohol and a thiol.

15. The fluid flow network of claim 1, wherein the coating comprises at least one protein selected from the group consisting of fibronectin, fibrin and fibrinogen.

16. The fluid flow network of claim 1, wherein the internal surface of the fluid duct comprises at least one selected from the group consisting of silica or glass, and wherein the coating comprises at least one silane.

17. The fluid flow network of claim 1, which is part of an organism's vasculature.

18. The fluid flow network of claim 17, wherein the fluid duct is an implantable device that is implanted within the organism's vasculature.

19. The fluid flow network of claim 17, wherein the organism comprises at least one selected from the group consisting of a plant and an animal.

20. A method of preventing or minimizing colonization of a fluid flow network comprising a fluid duct by a bacterium, wherein the fluid duct has a first opening and a second opening, wherein fluid can flow within the fluid duct from the first opening to the second opening, wherein the second opening comes into contact with the bacterium, the method comprising derivatizing at least a portion of the internal surface of the fluid duct with a coating that increases adhesion of the bacterium to the internal surface of the fluid duct as compared to the underivatized portion of the internal surface of the fluid.

21. The method of claim 20, wherein at least a portion of the internal surface of the fluid duct in proximity of at least one selected from the group consisting of the first opening and the second opening is derivatized with a coating that increases adhesion of the bacterium to the internal surface of the fluid duct as compared to the underivatized portion of the internal surface of the fluid.

22. The method of claim 20, wherein essentially the entire internal surface of the fluid duct is derivatized with a coating that increases adhesion of the bacterium to the internal surface of the fluid duct as compared to the underivatized internal surface of the fluid.

23. The method of claim 20, wherein the bacterium comprises an upstream surface migrating bacterium.

24. The method of claim 23, wherein the bacterium comprises Pseudomonas aeruginosa.

25. The method of claim 23, wherein the bacterium comprises at least one selected from the group consisting of Neisseria gonorrhoeae, Neisseria meningitidis, Legionella pneumophila, and Streptococcus sanguinis.

26. The method of claim 23, wherein the bacterium comprises at least one selected from the group consisting of Acinetobacter calcoaceticus, Aeromonas hydrophila, Azoarcus spp., Bacteroides ureolyticus, Branhamella catarrhalis, Comomonas testosterone, Dichelobacter nodosus, Eikenella corrodens, Kingella denitrificans, Kingella kingae, Legionella pneumophila, Moraxella bovis, Moraxella lacunata, Moraxella nonliquefaciens, Moraxella kingie, Mycoplasma mobile, Pseudomonas stutzeri, Pseudomonas putida, Pseudomonas syringae, Ralstonia solanacearum, Shewanella putrefaciens, Suttonella indologenes, Vibrio cholera, Wolinella spp., and/or Xylella fastidiosa.

27. The method of claim 20, wherein derivatizing the internal surface of the fluid duct in proximity of the second opening with a coating that increases adhesion of the bacterium to the internal surface of the fluid duct prevents or minimizes migration, or reduces the migration rate, of the bacterium through the fluid duct towards the first opening.

28. The method of claim 20, wherein the duct is part of at least one selected from the group consisting of a catheter, intravenous line and needle.

29. The method of claim 20, wherein the fluid flowing through the duct comprises a bodily fluid.

30. The method of claim 29, wherein the bodily fluid comprises at least one selected from the group consisting of blood, serum, plasma and urine.

31. The method of claim 20, wherein at least one selected from the group consisting of the first opening and second opening is in proximity to a branching point of the network, wherein the fluid duct is in fluid communication with one or more other fluid ducts.

32. The method of claim 31, wherein at least a portion of the internal surface of the fluid duct in proximity to the branching point of the network is derivatized with a coating that increases adhesion of the bacterium to the internal surface of the fluid duct as compared to the underivatized portion of the internal surface of the fluid.

33. The method of claim 20, wherein the coating comprises at least one chemical group selected from the group consisting of an alcohol and a thiol.

34. The method of claim 20, wherein the coating comprises at least one protein selected from the group consisting of fibronectin, fibrin and fibrinogen.

35. The method of claim 20, wherein the internal surface of the fluid duct comprises at least one selected from the group consisting of silica or glass, and wherein the coating comprises at least one silane.
